(19) United States
(12) Patent Application Publication
(10) Pub. No.: US 2014/0323589 A1
(43) Pub. Date: Oct. 30, 2014

(54) Method for the preparation of composite silica alcogels, aerogels and xerogels, apparatus for carrying out the method continuously, and novel composite silica alcogels, aerogels and xerogels

(71) Applicant: DEBRECENI EGYETEM, Debrecen (HU)

(72) Inventors: István Lázár, Debrecen (HU); István Fábián, Debrecen (HU)

(73) Assignee: Debreceni Egyetem, Debrecen (HU)

(21) Appl. No.: 14/354,249

(22) PCT Filed: Oct. 26, 2012
(86) PCT No.: PCT/HU2012/000115

§ 371 Date: Apr. 25, 2014

(30) Foreign Application Priority Data
Oct. 28, 2011 (HU) P1100603

(51) Int. Cl. B01J 31/02; G21F 1/02; A61K 47/02; F16L 59/02

(52) U.S. Cl. CPC B01J 31/0274; F16L 59/028; G21F 1/026; A61K 47/02
USPC 514/770; 502/150; 502/167; 502/168; 502/172; 502/159; 502/173; 502/5; 252/62; 252/478; 435/176; 422/224; 264/52

(57) ABSTRACT

The invention relates to a method for the preparation of composite silica alcogels, aerogels and xerogels, comprising
i) providing a reaction mixture comprising at least the following:
silane reagent,
base catalyst,
gelation retarding additive,
aqueous/organic solvent mixture,
guest particle,
ii) agitating the reaction mixture as necessary and sufficient until achieving the viscosity where the spontaneous movement of the guest particles does not occur anymore; and
iii) shaping the material obtained to a desired shape during or after step ii); then
iv) drying, if desired

The method according to the invention is also useful in continuous manufacturing technology, and the invention provides an apparatus for applying the method.

The invention further provides novel composite silica alcogels, aerogels or xerogels obtainable by the method according to the invention.

The invention and field of use thereof

The invention relates to a method for the preparation of composite silica alcogels, aerogels and xerogels, comprising using additives to change the viscosity of the reaction mixture according to a schedule. The additives are preferably compounds that do not react with the other components of the reaction mixture, and the molecules or molecular associations thereof are capable to form at least two hydrogen bonds at the same time.

The method according to the invention is also applicable to continuous manufacturing technology, and the invention relates to the apparatus for carrying out the continuous method.

The invention also relates to novel composite silica alcogels, aerogels and xerogels obtainable by the method according to the invention.

The composite silica alcogels, aerogels and xerogels produced according to the method of the invention are useful, in particular, in the following fields: preparation of catalysts, thermal insulation, thermal insulating radiation protection, medicine.

### BACKGROUND OF THE INVENTION

The silica aerogels are solids with the lowest density in the world, which are surprisingly strong in spite of the fact that 95% of their volume is air. The purest varieties are glass-like clear, thermally stable up to several hundred degrees of Celsius, and are the best heat and sound insulation materials in the world. They have huge specific area, their chemical composition may be varied greatly, therefore they are ideal candidates for the preparation of, for example, absorbents, gas and liquid filters, heterogeneous phase catalysts, as well as for the ultra lightweight heat and sound insulation of windows, buildings, vehicles.

The production of silica aerogels is done by sol-gel technology, during which the slow hydrolysis and polycondensation of usually a silane monomer or a prehydrolized silane oligomer in aqueous or aqueous/organic solvent results in a self-supporting silica gel framework (alcogel) that is dried with a suitable process (typically under supercritical conditions, or with freeze-drying) into an aerogel that retains the original mesoporous gel structure. (If the drying process is freeze-drying, then the aerogel obtained is also called as cryogel in the literature.)

Xerogels can be produced similarly to the aerogels, with the difference that the drying of the alcogel is carried out in a conventional way on air or in a drying chamber, rather than under supercritical conditions or with freeze-drying. Xerogels suffer significant constriction compared to aerogels during drying. The structure characteristic to the alcogels partially changes, resulting in smaller specific area, higher density, higher mechanical strength, and further they are not as good heat and sound insulators. Their field of application partially overlaps with that of the aerogels.

By including guest particles into the alco-, xero- and aerogels, composite alcogels, xerogels and aerogels can be obtained that have very diverse properties and fields of use.

The composite alcogels (in addition to the fact that composite xerogels and aerogels can be obtained therefrom) are themselves useful for example to carry out liquid or gas phase heterogeneous catalysis. A specific class of the composite alcogels and aerogels is the ones having proteins, enzymes, living cells included (immobilized) into the matrix, and those can be used for different purposes in biotechnology, molecular biology or cellular biology, without the matrix affecting their functions.

The main fields of use for composite aerogels: preparation of catalysts, thermal insulation, thermal insulating radiation protection, medicine.

The fields of use for composite xerogels are identical with that of composite aerogels, with the difference that their specific area is smaller, however their mechanical strength is higher.

In the preparation of composite alcogels, aerogels and xerogels, uniform dispersal of the guest particles is a challenge, because the guest particles tend to sediment, emerge, sort according to their size and density. Although there are techniques in the art to solve the problem, these are valid on narrow fields, and the dispersal of low density (below 0.98 g/cm³) guest particles, large sized (larger than 1 mm) guest particles, and especially of high density (above 5.5 g/cm³) and large sized (larger than 1 mm) guest particles is completely unsolved.

U.S. Pat. No. 6,492,014 relates to mesoporous composite silica gels and aerogels. According to the description, the guest particle is introduced into the pre-formed sol near (within 10, preferably 3 minutes) the gelation point. The guest particles thus dispersable are solid, their size is up to 1 mm, preferably 1 nm-100 μm.

Prevention of the sedimentation of ferroelectric microparticles was achieved by rapid cross-linking in near-weightlessness (under microgravitational conditions) on an airplane. (“Preparation of Nonlinear Optical Aerogels and Xerogels in Parabolic Flights”, Susanne Lisinski, Lorenz Ratke; Microgravity Sci. Technol (2008) 20:1-5).

According to the Chinese patent application published as CN101254449, nanofibres are dispersed in a sol with dispersing agent (e. g. Na-stearate) and then the solution is allowed to gel.

According the scientific publications “Role of Urea in the Preparation of Highly Porous Nanocomposite Aerogels”; M. F. Casula, D. Loche, S. Marras, G. Paschina, A. Corrias, Langmuir, 2007, 23 (7), 3509-3512, and “Structural study of highly porous nanocomposite aerogels”, Daniela Carta, Anna Corrias, Gavin Mountjoy, Gabriele Navarra, Journal of Non-Crystalline Solids 353 (2007) 1785-1788, urea is used in a double catalyzed alcogel (and aerogel) preparation method as gelation facilitating additive to produce a high porosity, magnetic aerogel nanocomposite.

The disadvantage of the above approaches is that they do not allow the dispersion of guest particles with very diverse physical properties (in particular, very low density materials, such as gases, and high density particles, such as heavy metals). Accordingly, there is a need for a method to allow the uniform dispersion of guest particles of any state of matter and density that are chemically composed of a single or multiple components, in silica alcogels, aerogels and/or xerogels.

A further disadvantage of the above approaches is that the procedures can be carried out in batches only; however, high-volume production requires a method usable in continuous operational mode.

The object of the invention is to avoid one or more of the above disadvantages. The present invention enables, on one hand, the dispersion of guest particles with different properties, and on the other hand, enables the use of the process in continuous manufacturing technology.

### BRIEF SUMMARY OF THE INVENTION

It was found that gelation can be slowed down during the preparation of silica alcogels and a long-lasting viscous region can be achieved by using certain additives, that facilitates, on the one hand, the dispersion of guest particles with different properties, and on the other hand, allows the use of the process in continuous manufacturing technology.

Based on the above, the present invention relates to a method for the preparation of composite silica alcogels, aerogels or xerogels, comprising

i) providing a reaction mixture comprising at least the following:

silane reagent,

base catalyst,

gelation retarding additive,

aqueous/organic solvent mixture,

guest particle,

ii) agitating the reaction mixture as necessary and sufficient until achieving the viscosity where spontaneous movement of the guest particles does not occur anymore; and

iii) shaping the material obtained to the desired shape during or after step ii); then

iv) drying, if desired.

The present invention further provides an apparatus to apply the above method in continuous manufacturing technology, said apparatus is provided with a 1 reagent vessel for receiving a silane reagent or a solution thereof and a 2 reagent vessel for receiving a solution of the base, a 3 reaction chamber, to which the 1,2 reagent vessels are connected, and a 4 mixing device having mixing elements positioned in the 3 reaction chamber.

The present invention further provides a composite silica alcogel, aerogel or xerogel, obtainable by the above method, and in which guest particles with density below 0.98 g/cm³ or with size over 1 mm are dispersed.

In the method according to the present invention, the gelation retarding reagent is preferably a compound that does not react with the other components of the reaction mixture, and the molecules or molecular associations thereof are capable to form at least two hydrogen bonds at the same time.

According to a preferred embodiment, there are at least two bridgehead atoms capable of forming hydrogen bonds in the molecule of the gelation retarding additive, wherein the distance between the closest bridgehead atoms is preferably up to 6 chemical bonds, as calculated on the shortest possible route on the covalent backbone of the molecule.

In a particularly preferred embodiment, the gelation retarding additive is urea, dimethylformamide, dimethyl sulfoxide or a diol, such as ethylene glycol or propylene glycol or a polyol, such as glycerol or cellulose or a mixture thereof.

In another embodiment, there is one bridgehead atom capable of forming hydrogen bonds in the molecule of the gelation retarding additive, and the molecules form molecular associates that are capable of forming at least two hydrogen bonds.

In the latter embodiment, the gelation retarding additive is preferably pyridine.

The base catalyst is preferably ammonia, ammonium carbonate, ammonium fluoride, hydrazine, hydroxylamine, or primary, secondary or tertiary amines, or a mixture thereof.

In a further embodiment, the gelation retarding additive plays the role of the catalyst as well, and is selected from the group consisting of the following: polyol amines, such as diethanolamine or triethanolamine, or di- or polyamines and amino alcohols, such as ethylenediamine, diethylenetriamine, diethanolamine, triethanolamine, piperazine, dibenzylamine, as well as diaza crown ethers containing two nitrogens and ether oxygens, for example 1,10-diaza-4,7,13,16-tetraoxacyclooctadecane, or a mixture thereof.

The silane reagent is preferably selected from the group consisting of the following: alkoxysilanes, prehydrolized alkoxysilanes, open-chain or cyclic alkoxysilane oligomers, alkylalkoxysilanes, arylalkoxysilanes, arylalkylalkoxysilanes, glycidoxypropylalkoxysilanes, halogenoalkoxysilanes, halogeonalkylalkoxysilanes, vinylalkoxysilanes, alkenylalkoxysilanes, alkynylalkoxysilanes, as well as other substituted alkoxysilanes, including carbon chain substituted derivatives thereof, or a mixture thereof.

In a particularly preferred embodiment, the silane reagent is tetramethoxysilane or tetraethoxysilane.

The gelation retarding additive amounts to preferably 1 to 50% of the reaction mixture.

The agitation of the mixture in step ii) of the method according to the invention is continued until reaching a viscosity of preferably about 2000 mPa·s.

The aqueous/organic solvent mixture is preferably an aqueous alcoholic mixture, in particular methanol-water mixture.

In an embodiment, a cosolvent is also used, which is preferably ethanol, isopropanol, propanol, acetone, t-butanol, i-butanol, n-butanol, ethylene glycol, propylene glycol, dimethyl-formamide and/or dimethyl sulfoxide.

The guest particle is preferably an element, alloy, an inorganic, organic, element-organic compound that does not react with the reaction mixture and is not or minimally soluble therein, composite, nanocrystal, nanorod, nanofilament, graphene, polymer, protein, enzyme, hormone, nucleic acid, fungus, spore, biological tissue, cell and/or virus, or a combination thereof.

In step ii), the agitation of the mixture is preferably carried out continuously or intermittently, by shaking, rotating the reaction vessel, by mechanic or magnetic or magneto-hydrodynamic mixing of the mixture, by migration of electrically or magnetically charged particles, by flowing the reaction mixture, by passing through a liquid or gas and/or by ultrasonic treatment, or a combination of the processes listed.

The extended embodiment of the apparatus according to the present invention further comprises a 5 particle tank for receiving at least one emulsion or suspension connected to a 4b mixing device, and/or a 6 macro chamber for receiving macroparticles and/or a 7 gas-forming chamber for gas or gas-forming reagent(s),

wherein the 1,2 reagent vessels are connected to 8a, 8b feeding devices connected to a 9 mixing chamber provided with a 4a mixing device,

wherein each of the 5 particle tank, the 6 macro chamber and the 7 gas-forming chamber are connected to 8c, 8d and 8e feeding means independently coupled either to said 9 mixing chamber provided with the 4a mixing means, or to a 9a second mixing chamber provided with a 4b mixing means,

the 9 mixing chamber is connected to the 3 reaction chamber provided with the 4 mixing device, the 3 reaction chamber is connected to the 9a second mixing chamber, and the 9a second mixing chamber is connected to a 3a second reaction chamber provided with a 4d mixing means.

### DESCRIPTION OF THE FIGURES

FIG. 1 shows the gel setting time as a function of the volume of the base catalyst.

FIG. 2 shows the width of the viscous region (expressed as the difference of the reaction times belonging to the 0.2 s and 10 s fall times) as a function of the volume of the base catalyst.

FIG. 3 shows the increase of gel setting time as a function of the volume of added urea, in the case of constant final volume composition.

FIG. 4 shows the change of viscosity, characterized with fall time as a function of the volume of added urea, in the case of constant final volume composition.

FIG. 5 shows the apparent gel setting time as a function of the volume of added DMSO additive and the ratio of TMOS/NH₃, in the case of constant final volume composition.

FIG. 6 shows the schematic of a continuous operating apparatus.

FIG. 7 shows the schematic of the continuous manufacturing process according the present invention that is suitable for the preparation of alcogel composites, alcogel foams and foamed alcogel composites comprising solid and/or liquid and/or gaseous particles dispersed alone or in combination.

FIGS. 8 to 35 show the pictures of composite silica alcogels/xerogels or aerogels prepared according to the present invention. Series “a” (8a, 9a . . . 35a) is the color photograph of the prepared products, series “b” (8b, 9b . . . 35b) is the black and white picture made from the color photograph (to enable black and white reproduction).

FIG. 8 shows a photograph of alcogels prepared with urea, the dispersed particles from left to right are the following: quartz sand, magnetite, glass beads, copper powder, iron powder, lead sand, iron(III)-oxide.

FIG. 9 shows a silica alcogel comprising dispersed oil droplets, made with urea additive.

FIG. 10 shows a picture of manually dispersed aerogel composites comprising heterogeneous phase materials, made with urea or DMF additive. The back row contains on the left an aerogel comprising calcium phosphate, hydroxyapatite and cellulose in combination, on the right an aerogel made with Cr₂O₃. The first row contains on the left an aerogel comprising lead sand, on the right an aerogel comprising yellow lead oxide. In the middle of the picture, there is an aerogel foam in which the larger pores are made by the dispersion of paraffin oil by the described method, then by leaching it out after the cross-linking of the alcogel, there are air filled bubbles within the aerogel in the place of the paraffin oil droplets.

FIG. 11 shows a microscopic picture (50× magnification) of an aerogel prepared from an alcogel comprising dispersed oil, made with urea additive.

FIG. 12 shows a silica alcogel comprising dispersed polystyrene foam beads, made with urea additive.

FIG. 13 shows a microscopic picture (20× magnification) of a piece of a composite aerogel comprising dispersed Cr₂O₃ powder, made with urea additive.

FIG. 14 shows a xerogel monolith obtained by slow, 5-day long atmospheric drying of an alcogel comprising calcium phosphate and microcrystalline cellulose, made with urea additive, showing significant constriction compared to its original size as shown by the mold.

FIG. 15 shows a microscopic picture (20× magnification) of a composite aerogel comprising lead oxide, made with urea additive.

FIG. 16 shows a picture of an aerogel comprising calcium phosphate, hydroxyapatite and cellulose.

FIG. 17 shows a picture of alcogels comprising lead sand (top left), iron powder (top right) and copper powder (bottom), made with DMF additive.

FIG. 18 shows a picture of an aerogel comprising lead sand, made with DMF additive.

FIG. 19 shows a microscopic picture (125× magnification) of a silica aerogel composite comprising lead sand, made with DMF additive.

FIG. 20 shows a picture of an aerogel comprising copper powder, made with DMF additive.

FIG. 21 shows a picture of an aerogel comprising iron powder, made with DMF additive.

FIG. 22 shows a picture of an alcogel comprising large glass beads, made with DMF additive.

FIG. 23 shows a picture of a silica aerogel composite comprising glass beads with 3-4 diameter, made with DMF additive.

FIG. 24 shows a picture of a cellulose aerogel, made with DMF additive.

FIG. 25 shows a picture of a cellulose aerogel, made with DMF additive, after calcination.

FIG. 26 shows an alcogel in a test tube, comprising dispersed air bubbles and polystyrene beads, made with DMSO additive.

FIG. 27 shows alcogel composites in test tubes, comprising materials with very different densities, made with DMSO additive. The evenly dispersed heterogeneous particles are: polystyrene beads and air bubbles on the left side, lead sand in the middle, and polystyrene beads and lead sand in combination on the right side.

FIG. 28 shows silica alcogels, comprising lead sand and polystyrene foam beads, made with or without the addition of DMSO additive.

FIG. 29 shows silica alcogel composites comprising high density particles, made with DMSO additive. On the left side, a piece of tin with length of 20 mm and diameter of 4 mm, in the middle, lead lumps with 6-8 mm diameter, fixed within the silica alcogel matrix. On the right side, there is a silica alcogel comprising lead sand and polystyrene foam beads in combination.

FIG. 30 shows a picture of a silica alcogel composite comprising dye-filled polypropylene beads, made with DMSO additive. The mean density of the beads is about 1.4 g/cm³.

FIG. 31 shows an alcogel in a 20 mm diameter glass tube, comprising dispersed nitrogen bubbles, made with DMSO additive.

FIG. 32 shows a picture of an aerogel, made with cellulose. The cellulose is the gelation retarding additive, dispersed particle and calcifiable pore-forming at the same time.

FIG. 33 shows a picture of an aerogel, made with cellulose, after calcification.

FIG. 34 shows alcogels comprising polypropylene granulate. These alcogel composites were prepared by compounds which serve the function of catalyst and gelation retarding additive simultaneously. The additives used from left to right are: diethylenetriamine, tetramethylethylene-diamine, piperazine, and 2,2′-(ethylenedioxy)-diethylamine.

FIG. 35 shows a picture of an alcogel comprising hydroxyapatite, made with urea additive, using the continuous technology. In the horizontally positioned alcogel, the guest particles are only in the bottom third of the sample due to running out of hydroxyapatite, the other five samples have uniform filling and distribution.

### DEFINITIONS

We use the following definitions in connection to the invention:

(Silica) alcogel: a gel formed by the hydrolysis and polycondensation of alkoxysilanes in a medium containing some kind of alcohol and water.

(Silica) solvogel: a gel formed by the replacement of alcohol with another solvent (for example acetone) in the alcogel. If the solvent is water, the solvogel is also called hydrogel.

(Silica) aerogel: a gel with open structure, obtained from an alcogel or solvogel by drying in supercritical medium, and maintaining the internal structure characteristic to the alcogel or solvogel, the pores are filled with air after drying. The porosity (i.e. the integrated volume of the pores expressed as a percentage of the full volume of the monolithic gel) of aerogels is higher than 50%.

(Silica) cryogel: an aerogel that is a gel with open structure, obtained from an alcogel or solvogel by removing the fluid medium in frozen state at decreased pressure with sublimation, which is frequently powder like in its appearance and maintains the internal structure characteristic to the alcogel or solvogel, the pores are filled with air after the drying process.

(Silica) xerogel: a material comprising an open network, obtained from an alcogel, solvogel or aquagel by completely evaporating the fluid medium found in it under normal conditions.

Reaction time: the time elapsed from the moment of mixing the solutions comprising the different reagents (hereinafter referred to as solutions “A” and “B”).

Gelation time (or gel-setting time): the time after which the polished steel measuring ball within the gel in the reaction vessel does not sink further and stops. The viscosity measurement technique used and the falling ball type viscometer are described in Example 1 in detail).

“t1” the fall time at the start of the viscous region.

Start of the viscous region (S): by measuring with the falling ball type viscometer, the reaction time for 5 cm fall path to reach “t1” fall time.

“t2” the fall time at the end of the viscous region.

End of the viscous region (E): by measuring with the falling ball type viscometer, the reaction time for 5 cm fall path to reach “t2” fall time.

The width of the viscous region (W): the difference between the end and start of the viscous region, i.e.: W=E−S

In the context of the invention, the terms “particle” or “guest particle” mean any particle that is chemically different and separated by a phase boundary from the components of the homogeneous reaction mixture.

### DETAILED DESCRIPTION OF THE INVENTION

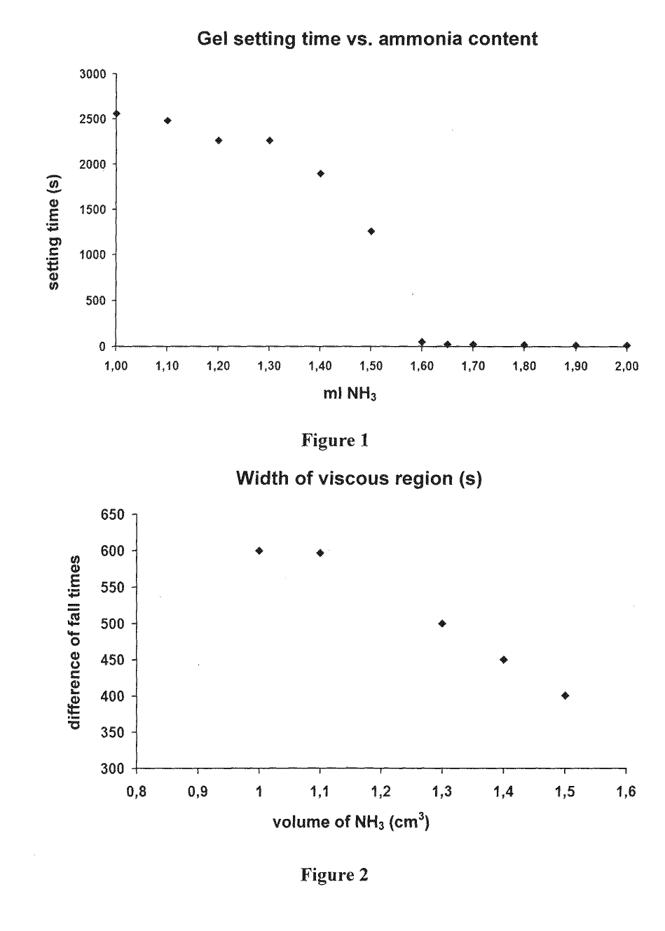

During studying the gelation of the alcogel formed by base catalyzed hydrolysis of tetramethoxysilane (TMOS), we unexpectedly found that certain additives (such as urea and ethylene glycol) significantly slow down the process of gelation, as well as of the process of the gel becoming self-supporting, while progressively enhance the viscosity of the reaction mixture (see Example 1, FIGS. 1 and 2).

This finding is even more surprising since M. F. Casula et al. and Daniela Carta et al. as cited above used urea as gelation facilitating additive in an alcogel (and aerogel) production process with double catalysis. In addition, U.S. Pat. No. 5,736,425 discloses that ethylene glycol accelerates the gelation carried out with ammonia catalyst.

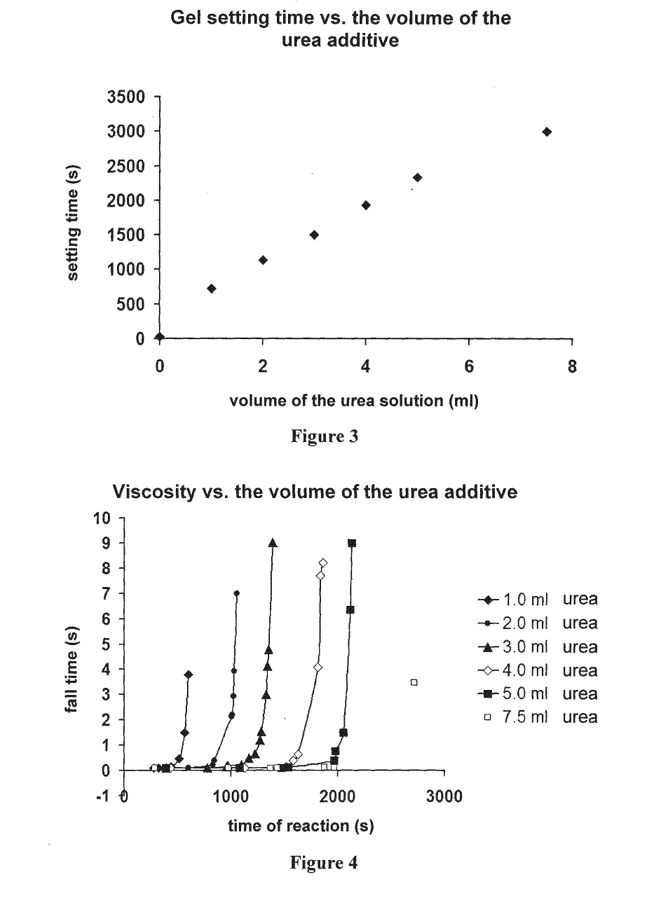

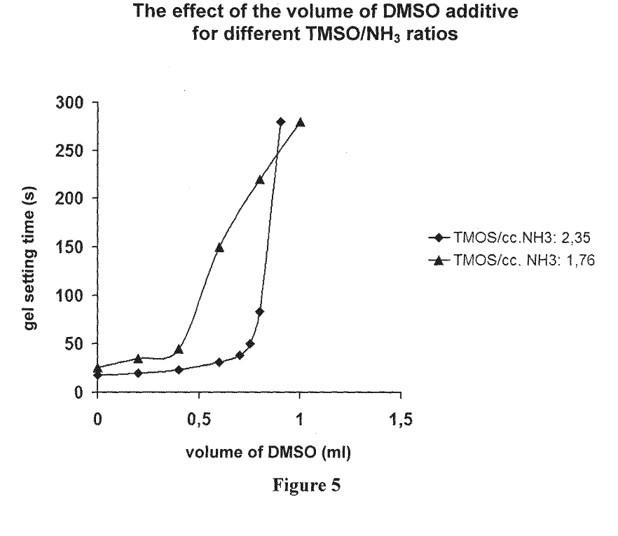

The gel-setting time can be controlled in a very wide range by changing the quantity and quality of the additives (see Example 1, FIGS. 3 and 4).

Based on our experiments, we think that the urea is capable to elongate the lifetime of the viscous region due to the fact that it forms hydrogen bonds similar to water with the Si—OH groups, therefore hinders the accessibility (and thus the condensation reaction) of the Si—OH groups to each other.

Accordingly, we carried out experiments with further substances, and surprisingly found that the effect can be achieved with compounds or molecular associations (such as diols, polyols, dimethylformamide, dimethyl sulfoxide) that are capable of forming multiple hydrogen bonds, while with simple alcohols that have only one OH group capable of forming such type of bonds, no advantageous results were achieved. Especially good results were obtained, in addition to urea, in the case of dimethylformamide, dimethyl sulfoxide, ethylene glycol, propylene glycol, glycerol and cellulose. The specific experimental results are partially described later (see Example 2).

The molecule of the additive contains at least two atoms that are capable to participate in a hydrogen bond as donor and/or acceptor (hereinafter referred to as: bridgehead atom). Bridgehead atoms can preferably be the following atoms: O, N, C, S, F, P, Cl. A bridgehead atom is considered as hydrogen bond donor if a hydrogen atom is bound thereto, and the hydrogen atom being part of the bond has a partial positive charge. A hydrogen bond donor bridgehead atom may also be a hydrogen bond acceptor, if it has a non binding electron pair. A bridgehead atom is considered exclusively as hydrogen bond acceptor, if no hydrogen atom binds thereto and has at least one non binding electron pair.

The distance (as calculated on the shortest possible route on the covalent backbone of the molecule) between the closest bridgehead atoms within the additive molecule is preferably no more than 6 chemical bonds.

Those additives are especially preferred in which the distance of the bridgehead atoms is 1-4 chemical bonds.

Those additives are the most preferred in which the distance of the bridgehead atoms is 1-3 chemical bonds.

Similarly to the above situation, those additives can also be used whose molecules although only contain a single bridgehead atom, but who are present in the form of molecular associations that have at least two bridgehead atoms, therefore are also capable of forming multiple hydrogen bonds. The binding force between the molecular associations may be for example π-π stacking interaction or hydrogen bond.

The distance between the bridgehead atoms within the molecular associations is preferably up to 15 Å.

Specific examples for the additives containing at least two bridgehead atoms are, among others, urea, dimethylformamide, dimethyl sulfoxide, diols, such as ethylene glycol, polyols, such as glycerol or cellulose.

One example for the additives containing one bridgehead atoms is pyridine, the molecules of which form associations by π-π stacking interaction, which show similar behavior to the diamines.

There are compounds that, in addition to be capable of forming at least two hydrogen bonds, also contain a basic moiety, therefore they can play the role of the base and gelation retarding additive at the same time. Such are the amines containing several OH groups, such as diethanolamine and triethanolamine and similar compounds with polyol structure, the compounds containing several amino groups, such as diethylenetriamine and piperazine, or the open-chain or cyclic compounds containing ether oxygens and amine groups, for example 2,2′-(ethylenedioxy)-diethylamine or 1,10-diaza-4,7,13,16-tetraoxacyclooctadecane.

The reaction mixture, created with the suitable additives and/or the necessary amount of base catalyst, having the progressively increasing viscosity, until it finally solidifies, enables the dispersion of heterogeneous phase guest particles therein, thus preparing composites.

In the following, the method according to the invention for the preparation of composite silica alcogels, aerogels and xerogels is described in more detail.

Silane Reagent

The silane reagent useful in the method according to the invention alone or in combination with other silane reagents, in particular with TMOS, are other alkoxysilanes (tetraethoxysilane, among others), prehydrolized alkoxysilanes, open-chain or cyclic alkoxysilane oligomers, alkylalkoxysilanes, arylalkoxysilanes, arylalkylalkoxysilanes, glycidoxypropylalkoxysilanes, halogenoalkoxysilanes, halogeonalkylalkoxysilane, vinylalkoxysilanes, alkenylalkoxysilanes, alkynylalkoxysilanes, as well as other substituted alkoxysilanes (among others cyclohexyltrimethoxysilane, phenyltrimethoxysilane, dimethyldimethoxysilane, glycidoxypropyltrimethoxysilane, vinyltrimethoxysilane).

It is not necessary to dissolve the silane reagent or the mixture thereof in a solvent, it can be added in solvent-free form during the reaction. The use of a solution is expedient due to the manageability aspects (smoother feeding), but it is not mandatory.

The silane reagent or the solution thereof is also referred to as solution “A” throughout the description.

Catalyst

The method according to the invention is carried out in the presence of a base catalyst. The base catalyst may be, among others, an organic or inorganic amine, in particular ammonia, ammonium carbonate, ammonium fluoride, hydrazine, hydroxylamine, or primary, secondary or tertiary amines. Further, di- and polyamines and amino-alcohols are useful as catalyst, such as ethylenediamine, diethylenetriamine, diethanolamine, triethanolamine, piperazine, dibenzylamine, as well as diaza crown ethers containing two nitrogens and ether oxygens, for example 1,10-diaza-4,7,13,16-tetraoxacyclooctadecane. Obviously, carbon chain substituted variants of these may also be used as catalyst.

As mentioned above, there are compounds that may play the role of the catalyst and gelation retarding additive simultaneously, these are for example polyol amines, such as diethanolamine or triethanolamine, or di- or polyamines and aminoalcohols, such as ethylenediamine, diethylenetriamine, diethanolamine, triethanolamine, piperazine, dibenzylamine, as well as diaza crown ethers containing two nitrogens and ether oxygens, for example 1,10-diaza-4,7,13,16-tetraoxacyclooctadecane.

The most widespread catalyst is ammonia, in particular an aqueous ammonia solution of 10-25%. The amount of the ammonia catalyst—by using 25% ammonia solution diluted 1:1 by volume—is typically 5-25% v/v, preferably 10-15% v/v, based on the volume of the reaction mixture without the heterogeneous phase additives.

The catalyst or the solution thereof is also referred to as solution “B” throughout the description.

The Reaction Medium

With respect to the solvents used, the silane reagent—if used in the form of a solution—is dissolved in a non-aqueous solvent, the base catalyst is dissolved in water or in an aqueous-organic solvent mixture. Generally an alcoholic solvent is also used for at least one of the solutions. The most frequently used alcohol is methanol. By mixing the two reagent solutions, an aqueous-organic, most frequently aqueous-alcoholic mixture is formed that is serving as the reaction medium.

The reaction mixture is therefore an aqueous-organic mixture, generally an aqueous-alcoholic mixture, particularly preferably a methanol-water mixture. The alcohol-type co-solvent not increasing the gelation time may be ethanol for preparing transparent (optical) gels, while isopropanol, propanol, acetone, tert-butanol, i-butanol, n-butanol result in an alcogel with opaque or white, occasionally precipitated character. Opalescency is not a hindrance for practical purposes, except for optical ones.

Gelation Retarding Additive

As described in detail above, the gelation retarding reagent is a compound that does not react with the other components of the reaction mixture, and the molecules or molecular associations thereof are capable to form at least two hydrogen bonds simultaneously.

The additives for increasing the gelation time may serve as a co-solvent in certain cases, thus may facilitate the full dissolution of the further silane reagents (such as hexadecyltrimethoxysilane) used in addition to TMOS. Such additives serving as co-solvents may be for example ethylene glycol, propylene glycol, dimethylformamide, dimethyl sulfoxide, which are also useful for the preparation of transparent optical aerogel matrices.

The amount of gelation retarding additive depends on the quality of the additive itself and of the other reactants, the composition of reaction medium, and the intended gelation time; it generally makes up 1-50% w/w, preferably 1-25% w/w of the reaction mixture.

In the method for the preparation of alcogel composites, it is preferred, but not mandatory, to use additives from the possible ones that are washed out spontaneously after the formation of the alcogel during the solvent exchange processes, i.e. that are easily removable. Particularly preferred are the additives that are generally used as organic solvents themselves in other applications. If the alcogel after preparation goes through high temperature treatment during further processing, then it is expedient and reasonable to use partially soluble or not soluble additives (such as cellulose powder). In this case the additive burns out during the heat treatment, and holes remain in its place, therefore macroporous, porous or spongy aerogels may be produced.

Guest Particle

With the additive aided viscosity increasing method, particles having individual size from nanometer to several millimeters may be kept dispersed, the density of which extends to the physically attainable full density range, it is not sensitive whether very low or very high density particles are used, and equally useful for a set of particles composed of a single or multiple materials and having single or multiple densities. The solid particles are added to the in situ formed reaction mixture together with the components of the reaction mixture in dry form, or in the form of a suspension made with a solvent miscible with the reaction mixture (preferably the same as used therein) or a suspension with a liquid non miscible therewith. The particles (if their nature permits it) may be admixed into any of the reagent solutions. In other cases, an emulsion of liquids, or a gas having the necessary bubble size and dispersed with the necessary mixing, or a gas-forming reagent, or a a volatile gas-forming material may be added. To create gas bubbles, a method may be used wherein a gas physically soluble in the reaction mixture is absorbed in the reaction mixture under pressure (for example by adding the reaction mixture into a reactor with appropriate atmosphere and placing it under pressure), then after reaching high enough viscosity, but before gel-setting, the pressure is dropped to ambient value within a short period of time.

The method according to the invention is universally useful in the case of liquid or gaseous phase particles, the liquids including suspensions and emulsions.

In the context of the invention, the terms “particle” or “guest particle” mean any particle that is chemically different and separated by a phase boundary from the components of the homogeneous reaction mixture.

The particle therefore may be colloid particle or powder with various fineness, crystalline or amorphous or glass-like particulate solid, polymer, liquid, organic or inorganic gel, organic or inorganic foam, emulsion, suspension, gas bubble.

According to the present invention, the guest particle is preferably an element, alloy, inorganic, organic, element-organic compound that does not react with the reaction mixture and is not or minimally soluble therein, composite, a material organized in space, plane or line lattice in its local crystal structure, macromolecule, polymer, protein, enzyme, hormone, nucleic acid, fungus, spore, biological tissue, cell and/or virus, or a combination thereof.

The method according to the invention is applicable to any particle with any density (according to the current state of science, the lowest and highest density materials, based on the density of the standard state hydrogen gas and osmium, 8.16·10⁻⁵-22.59 g/cm³) and with any form (liquid drop non-miscible with the reaction medium, gas bubble, nanoparticle, nanofilament, nanotube, nanosheet (e.g. graphene), regular or irregular particulate, crystal, filament, fiber, tissue). The method is also applicable to the dispersion and encapsulation of foams, foamed polymers, and particles of organic and inorganic gels. The inclusion/creation of liquid particles may be done similarly to the solid particles, or by subsequent admixing and dispersion in the reaction mixture, or by in situ synthesis by chemical reaction, or by mixing the reaction mixture with a heterogeneous liquid phase. The generation of gas bubbles may be done by injecting a gas under pressure into the reaction mixture through a surface with appropriate porosity or through a capillary system (with or without the addition of agents modifying the surface tension), by aspiration through the same system under reduced pressure, by evaporating the volatile components of the system at reduced pressure and/or increased temperature as necessary (by abruptly foaming or boiling in vacuum), or by in situ gas production, wherein the gas production technique may be some kind of chemical reaction, expansion at ambient pressure of a gas (e.g. N₂, CH₄) dissolved at a higher pressure than the outside pressure, liberation of the atmospheric gases dissolved due to the mixing of the components of the reaction mixture, or by injecting a low boiling point liquid and in situ evaporation thereof (e.g. propane).

Mixing of the Components

For the prepared regent solutions to carry out the method, the condition must be met that the silane reagent solution may not be in contact with the water and the catalyst. The other components, i.e. the additive and guest particles may be admixed to any of the reagents solutions or may be added to the reaction mixture from a separate container. In the case of the guest particles, based on the type of the particle, the addition from a separate container may be advantageous from an operational standpoint. If a gelation retarding additive is used that is a co-solvent to facilitate the dissolution of the silane reagent, it is expedient to add it to the solution of the silane reagent.

Gel Forming Reaction

The reaction may be conveniently carried out at room temperature and atmospheric pressure (20-35° C., 800-1080 hPa).

The time of reaction may be regulated by the quantity, quality of the additive used, the quantity of the catalyst used, the quantity and concentration of the reactants. The composition necessary to achieve the desired reaction time may be determined by simple experiments.

The length of the viscous region during the gel formation reaction may be regulated by the quantity, quality of the additive used, the quantity of the catalyst used, the quantity and concentration of the reactants.

The width of the viscous region (W) depends on the composition of the reaction mixture, on the quantity of the additive, and by definition, on the values t1 and t2, wherein “t1” is the fall time associated with the beginning of the viscous region, and “t2” is the fall time associated with the end of the viscous region.

In FIGS. 1 to 5, t1=0.2 s. This corresponds to about a viscosity of 5 mPa·s.

According to the present invention, t2 is preferably 1 s≦t2≦3600 s, more preferably 1 s≦t2≦600 s, and most preferably 2 s≦t2≦60 s. In FIGS. 1 to 5, t2=10 s. This corresponds to about a viscosity of 2000 mPa·s.

According to the present invention, when t1=0.2 s and t2=10 s, the width of the viscous region (W) is preferably 10 s≦W≦7200 s, more preferably 10 s≦W≦3600 s, and most preferably 30 s≦W≦3600 s.

In other words: the length of the viscous region (W) is preferably 10-7200 s, more preferably 10-3600 s, and most preferably 30-3600 s, i.e. the viscosity of the reaction mixture is preferably kept for this time on a value that is advantageous to the dispersion of the guest particles.

The gelation time is typically set between 10 and 120 minutes to enable the appropriately fine distribution of the particles, but especially in the case of continuous technology, shorter times may be used depending on the construction and length of the apparatus.

The reaction mixture, formed by the appropriate additive and/or necessary amount of base catalyst, that has gradually increasing viscosity until it finally solidifies, enables—by continuous or intermittent agitation, such as by shaking, rotating the vessel containing the reaction mixture, mixing the reaction mixture by mechanical or magnetic or magneto-hydrodynamic means, migrating electrically or magnetically charged particles, flowing the reaction medium, bubbling through a liquid or a gas, ultrasonication, or by a combination of the listed techniques—the dispersion of heterogeneous phase particles therein (without a change in the size of the particles), dispersing larger particles (e.g. non-miscible liquid phase materials, or suspensions or emulsions thereof, or suspensions or emulsions comprising solvents soluble in the reaction mixture) into smaller sizes, or generating gas bubbles by in situ chemical reaction or physical technique without the release or fusing thereof. During the above described necessary and sufficient agitation of the reaction mixture, the sedimentation/sorting/mergence/emergence of the particles forming the heterogeneous phase within the reaction mixture considered as homogeneous does not continue after a certain time due to the fast increase in viscosity, therefore the heterogeneous phase particles with various size and density are fixed into the alcogel formed through cross-linking. The term necessary and sufficient agitation means that the extent of agitation is sufficient to keep the distribution uniform, but it is not too strong, i.e. does not destruct the structure of the gel during its formation. Setting up the appropriate agitation is a routine task for the person skilled in the art. The method is suitable for the simultaneous, uniform dispersion of particles having very large density difference (for example air bubbles, lead particles and polystyrene foam) in the silica alcogel.

During the formation of the gel, the reaction mixture is agitated until the particles in the forming pre-alcogel are practically fixed in their position, but the pre-alcogel is still fluidic with very high viscosity (accordingly, the viscosity is strongly dependent on the particle size and density of the dispersed particle, usually is over 2000 mPa·s, but can be even about 100000 mPa·s), plastic and appropriately malleable.

Shaping

The pre-alcogel obtained as described above is formed into the desired shape by allowing the completion of the gelation to occur immediately after shaping, or during the end stage of shaping.

According to another aspect, shaping, and in particular molding may be carried out during the gel formation reaction, prior to the guest particles being practically fixed in the forming pre-alcogel. In this case, the selective agitation of the reaction mixture and/or dispersed guest particles may be continued after shaping, for example within the mold.

Preparation of Xerogels and Aerogels

From the alcogels obtained as described above, (after chemical functionalization, for example after surface siliconizing to make it hydrophobic as necessary) after the optionally necessary solvent exchanges, by drying with the evaporation of the liquid solvent, xerogels may be obtained, and by drying under supercritical conditions or by freeze drying, aerogels, aerogel based composites or aerogel foams may be obtained.

Continuous Technology

It was unexpectedly found that in the method according to the present invention as described above, due to the possibility of the simultaneous and parallel introduction of the reaction components and the particles (in the broadest sense of the term), as well as of the regulation of the viscosity and gel-setting time within a wide range, the present method may be used, contrary to prior methods for the preparation of alcogels, not only in batch operation (i.e. intermittently), but in a continuous manufacturing technology as well, after appropriately setting up the blending/mixing/dispersing/generating process, gel-setting time and the width of the viscous region, by ensuring the continuous feeding of the reaction components and the appropriate mixing intensity and type.

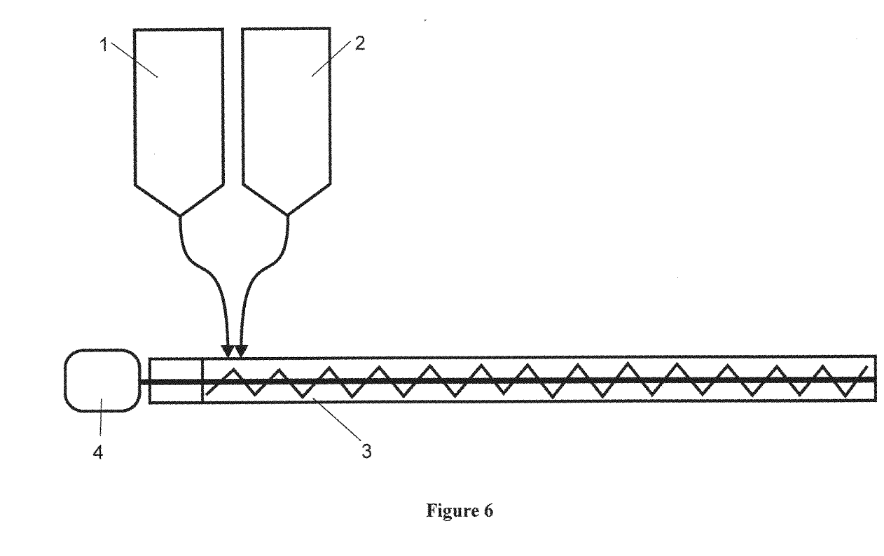

FIG. 6 shows a specific embodiment of the apparatus suitable for performing the continuous manufacturing technology.

This embodiment of the apparatus according to the invention contains two 1, 2 reagent vessels, one for receiving a silane reagent or a solution thereof and the other for receiving a base catalyst or a solution thereof, further, any one of the 1, 2 reagent vessels contains the additive and any one of the 1, 2 reagent vessels contains the guest particles. The apparatus further contains a 3 reaction chamber and a 4 mixing device having mixing elements positioned in the 3 reaction chamber.

In the arrangement shown in FIG. 6, feeding of the components occurs from the 1, 2 reagent vessels to the 3 reaction chamber. The 4 mixing device provides the homogeneous blending of the components at the front of the reaction chamber. During advancement, the viscosity of the reaction mixture continuously increases while the separation of the particles is inhibited by constant, regulated mixing as necessary using the 4 mixing device.

The reagent chamber in the simplest case is a gravitational feeding device (e.g. dropping funnel), in this case there is no need for a separate feeding device. The reaction chamber may be, but not necessarily, a tubular reactor.

The length of the 3 reaction chamber, the concentration of the reagents and particles fed thereto, the rate of feeding are harmonized so that the particles within the pre-alcogel leaving the 3 reaction chamber are in a practically fixed state, but the pre-alcogel is still fluid with very high viscosity, appropriately malleable and plastic, and is suitable to be formed into the desired shape by allowing the completion of the gelation to occur immediately after shaping, or during the end stage of shaping.

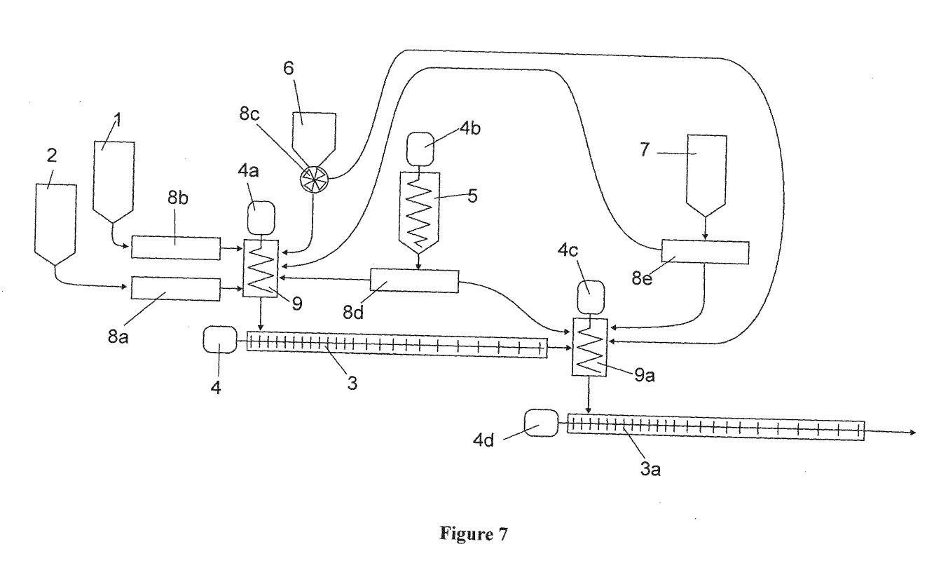

FIG. 7 shows another specific embodiment of the apparatus according to the invention that is complemented with several optional elements.

The guest particles may be introduced, on the one part, in the form of an emulsion or suspension, and on the second part, in solid form (especially in the case of the larger, so-called macroparticles), and on the third part, in the case of when gases are to be dispersed, in the form of gas-forming reagents. It is expedient to place these different forms into different vessels, therefore the apparatus may include, depending on the type of the particles to be introduced, a 5 particle tank for receiving at least one emulsion or suspension, a 6 macro chamber for receiving macroparticles, and/or a 7 gas-forming chamber for receiving gas or gas-forming reagent(s), which may be gas cylinder or a chamber containing gas-forming reagents, from which the feeding is performed through 8c, 8d, 8e feeding means. The 5 particle tank containing the suspension or emulsion is optionally mixed with an independent 4b mixing device to form and/or maintain the suspension or emulsion.

In the arrangement shown in FIG. 7, feeding of the reagents occurs from the 1, 2 reagent vessels and the 5 particle tank, 6 macro chamber and/or 7 gas-forming chamber independently with the help of the 8a, 8b, 8c, 8d, 8e feeding means (e.g. piston, centrifugal or peristaltic pumps, hydraulically sealed or ordinary screw, conveyor, cup, circular plate or other type feeders, etc.) that are working suitably harmonized, optionally regulated, but simple gravitational feeders (e.g. dropping funnel) may be used as well. The components of the reaction mixture are fed into the 3 reaction chamber mixed with the 4 mixing device. The reaction chamber may be, but not necessarily, a tubular reactor, the position of which may be horizontal, tilted or vertical. In a preferred embodiment, the reaction chamber is a slightly (at about)5-10°) tilted rotating tubular reactor, and near the inside surface of the reaction chamber, scraping means (not shown on the figure) are arranged to facilitate the removal of the mixture from the wall of the reactor.

At the front of the 3 reaction chamber, a homogeneous reaction mixture is formed, then this advances through the 3 reaction chamber. During advancement, the viscosity of the reaction mixture continuously increases while the separation of the particles is inhibited by constant, regulated mixing as necessary using the 4 mixing device. The length of the 3 reaction chamber, the concentration of the reagents and particles fed thereto, the rate of feeding are harmonized so that the particles within the pre-alcogel leaving the 3 reaction chamber are in a practically fixed state, but the pre-alcogel is still fluid with very high viscosity, malleable and plastic, and is suitable to be formed into the desired shape by allowing the completion of the gelation to occur immediately after shaping, or during the end stage of shaping.

The apparatus optionally may contain several 3, 3a reaction chambers, like a first 3 reaction chamber and a second 3a reaction chamber. The length of the 3, 3a reaction chambers, the concentration of the reagents and particles fed thereto, the rate of feeding are harmonized so that the mixture leaving the first 3 reaction chamber is in an already quite thick state, but is still far from the gelation point (at this point, the viscosity is below 1000 mPa·s, typically a few hundred mPa·s), and the particles within the pre-alcogel leaving the secondary 3a reaction chamber are in a practically fixed state, but the pre-alcogel is still fluid with very high viscosity (the viscosity associated with this state is highly dependent on the particle size and density of the dispersed particle, usually above 2000 mPa·s, but it can be up to about 100000 mPa·s), malleable and plastic, and is suitable to be formed into the desired shape by allowing the completion of the gelation to occur immediately after shaping, or during the end stage of shaping.

The use of two 3, 3a reaction chambers is particularly advantageous when we intend to introduce particles (either alone or in in addition to other particles) that are easier to mix into an already viscous mixture. An example of this is the feeding of a low-boiling liquid, which inflates the reaction mixture into a foam, or the admixture of an oil. To avoid the fusion of the bubbles or droplets, it is advantageous if the admixture occurs after passing through the first 3 reaction chamber, at the front of the 3a second reaction chamber into an already high viscosity mixture. A further example is the case when in addition to finer particles (which may be added into the first 3 reaction chamber) extremely high density and large particles are mixed in. It is not expedient to mix the latter particles immediately into the reaction mixture, because although this is possible, the handling of the mixture thus obtained is difficult. If it is possible, this operation should be postponed when the viscosity of the reaction mixture is already higher.

In order to homogeneously blend the reactants and the particles, it is expedient to insert an appropriately formed, suitably mixed primary 9, 9a mixing chamber in front of the 3, 3a reaction chamber. This will enable to accomplish the homogenization by another, typically higher intensity mixing than which is used in the 3, 3a reaction chamber. The use of the 9, 9a mixing chamber is particularly advantageous when the gelation time used is short, therefore the lack of sufficient mixing would result in premature gelation locally. At the same time, the too fast mixing could ruin the structure of the gel at the subsequent phase of the reaction.

The 9, 9a mixing chambers are mixed with the 4a, 4c mixing devices, while the a 3a reaction chamber is mixed with the 4d mixing means.

The 3, 3a reaction chambers may be integrated into the 9, 9a mixing chambers, or may be separated from them by varying length of tubes. In lack of the optional 9a second mixing chamber, the first and second 3, 3a reaction chambers may be integrated, or separated by a varying length of tube, as well as the presence of the 3a second reaction chamber is optional, or it may follow the primary mixing chamber.

The method of the present invention enables the generation of various shaped bricks, sheets, three-dimensional shapes with molding, pressing, extrusion or other technology, as well as further processing of the shapes obtained, such as dividing, cutting, etc. The continuous technique enables the use of the method in mass prodction. With respect to the reaction partners and guest particles, the above described details are valid.

The method according to the invention enables the dispersion of the particles mentioned, as necessary for practical applications.

The alcogels obtainable by the method according to the invention (batch or continuous) and the aerogels and/or xerogels prepared therefrom are especially useful in the following fields:

As support for catalysts or as catalysts in the case of particles with any density. Since the method according to the present invention enables the dispersion of particles with both extremely high and low densities, it is possible to create aerogel based catalysts in which the dispersed high density material is catalytically active (such as, for example, PbO, Fe₂O₃, CoO, V₂O₅, Cr₂O₃, Pd, Ni, Ag, nano-Au, Pt-colloid), and in which it is desired to provide a spongy structure in order to enhance the penetrability and accessibility by using gaseous, solvent-leachable or calcifiable additive in the aerogel (such as paraffin particles, paraffin oil, corkwood granulate, crystalline cellulose, polyurethane beads, polystyrene beads, as well as air, nitrogen, argon, etc.). The alcogel still contains both dispersed materials, and after supercritical drying, then the subsequent optional calcification, holes will remain in the place of said additive.

In the case of particles containing elements with high atomic number (such as Pb, Bi), radioactivity protection or elementary particle radiation protection materials may be produced that have lower specific density, and at the same time having heat resistant, heat insulating and sound insulating properties.

Electromagnetic shielding, electromagnetic absorber and wide spectral range black body having heat resistant, heat insulating and sound insulating properties.

Production of materials or metamaterials containing naonosized dispersed particles suitable for the alteration of the phase of electromagnetic radiation.

Aerogel composites and nanocomposites containing particles transparent in the visible region but absorbing or reflecting in the infrared region which enhances heat insulation.

Production of heat insulating windows absorbing in the UV region and transparent for visible light.

Heat insulation elements and vacuum insulation panels (VIPs) containing aerogel foams and having increased efficiency compared to aerogels.

In medical biology fields, such as production of compositions suitable for artificial bone replacement and bone regeneration, or support matrix for tissue growth.

Alcogels, aerogels or xerogels to immobilize cells, cellular components, bacteria, fungi, spores, pollens or viruses, useful in biotechnology, cell culture, medicine.

The following non-limiting examples further illustrate the present invention.

### EXAMPLES

### Example 1

### Study of the Gelation Retarding Effect of Urea

During studying the gelation of the alcogel formed by base catalyzed hydrolysis of tetramethoxysilane (TMOS), we unexpectedly found that in the constant volume reaction mixture the time for the alcogel becoming self-supporting does not decrease linearly with the amount of the base catalyst added, as expected, but it changes as shown in FIG. 1, as a function of the volume of the base catalyst added (FIG. 1).

In these studies, the base catalyst was 1:1 v/v diluted 25% NH₃ solution.

The term “constant volume” in these studies means that the sum of the volumes of the methanol used as solvent and the silane reagent (e.g. tetramethoxysilane, abbreviated: TMOS), and the volume of the water and the 1:1 diluted ammonia solution was usually constant both pair-wise and combined, typically 15 ml.

In a typical series of experiments, the following solutions were used in the studies. Solution “A”: 7.50 ml methanol, 0.80-0.90 ml water, 1.70-1.60 ml 1:1 diluted NH₃ solution, Solution “B”: 3.50 ml methanol, 1.50 ml TMOS. Solution “A” and “B” were blended at the time point t=0 by mixing with a magnetic stirrer, it was quickly homogenized by shaking the sealed vessel vigorously, then left to stand in a rack.

The measurement of viscosity was performed with a custom built falling ball viscometer type instrument. Compared to the factory built viscometers, it was an essential change in the structure of the instrument that the measurement ball did not fit quite tightly into the measurement tube, but it was surrounded by wide open spaces on the sides. This modification is necessary because in the higher viscosity region, the amount of the gel carried by the measurement ball hinders the movement, and shows gel-setting in the tight walled instrument already when the falling time, and consequently the viscosity characterized by the fall time, is still measurable in the wide measurement tube on a given fall distance. The steel ball used for the measurement (diameter=4.80 mm, density=7.56 g/cm³) is maneuvered from the outside by a magnet, and prior to reaching the first label, about 2 cm fall path was still ensured to achieve a constant speed. By releasing the steel measurement ball, we measured the time necessary to travel the exactly 5.00 cm free fall path between the top and bottom labels (fall time) as a function of reaction time. Based on the Stokes' law, the fall time multiplied with a constant that is characteristic for the instrument and the medium tested (in the present case, with constant composition) is directly proportional to the viscosity, therefore the fall time may be used for the characterization of the changes in viscosity (as well as to indicate the relative viscosity). For the precise measurements the experiments were recorded on video, and the elapsed reaction time and fall times were determined based on those. The fall times measured (due to the increasing viscosity during the measurement) were assigned to the reaction time required to reach the bottom label, which resulted in better correlation than with the reaction time required to reach the top label, or with the average of the reaction times associated with reaching the top and bottom labels.

Gel-setting time is the time after which the steel measuring ball with the polished surface (diameter=4.80 mm, density=7.56 g/ cm³) within the gel does not sink further in the reaction mixture, but stops. FIG. 1 clearly shows that at the higher added base volumes (1.60-2.00 ml) the gel-setting occurs with nearly identical speed. With slightly decreasing the amount of the base compared to the previous one, within a very narrow volume range (1.50-1.60 ml), an exponential type increase of the gel-setting time occurs, which coincides with the increase of viscosity at first moderately, then gradually and finally rather rapidly. Further decreasing the amount of the catalyst, the time for the gel becoming self-supporting can be approximated by a saturation type curve.

The lifetime of the viscous region (time width, W) may be regulated by the amount of the base catalyst. It is visible in FIG. 2—which shows the width of the viscous region (expressed as the difference of the reaction times associated with 0.2 s and 10 s fall times) as a function of the volume of the base catalyst—that between the volumes of 1.10-1.50 ml the width of the viscous region is inversely proportional to the amount of the base catalyst. During the hydrolysis of the silane reagent, a viscous region appears that is sustained in time with gradually increasing viscosity, the viscous fluidity of which is retained for a longer time (FIG. 2).

Based on the section of the curve shown in FIG. 1 corresponding to catalyzer volumes between 1.0-1.6 ml, there is a theoretical possibility, by the appropriately precise addition of the base, for the simultaneous, sensitive and non-linear regulation of the viscosity of the reaction mixture, of the width of the viscous region, and of the gel-setting time.

It follows from FIGS. 1 and 2 that by changing the amount of ammonia, a composition may be defined that is slow to cross-link and has a continuously increasing viscosity to facilitate the dispersion of guest particles. A very small change in the amount of ammonia within this range, such as the effect of the precision of measurement (especially at the standard volume measurements shown in FIG. 2, with 1.00-1.60 ml NH₃), has a very critical effect on the gelation time, therefore the fluctuation of the measurements makes the use of this steep region of the curve completely unreliable and unmanageable.

During our experiments performed in the above mentioned steep region of gelation, as well as with catalyst volumes above 1.60 ml shown in FIG. 1 (in the region with quick gel-setting) we unexpectedly found that certain additives (such as urea and ethylene glycol) significantly slow down the process of gelation, as well as of the gel becoming self-supporting during the base catalyzed hydrolysis of TMOS, while progressively enhance the viscosity of the reaction mixture. Therefore it is not necessary to work in the above mentioned uncertain region, but we can use the ammonia amounts over 1.60 ml shown on the right hand side flat region of FIG. 1. The gel-setting time within this region is very short without the additive, insensitive to the measurement precision. With the aid of the additive, the gelation time and viscous region is increased compared to this, but due to the additive, it can be well regulated.

The gel-setting time can be controlled in a very wide range by varying the quantity and quality of the additive, as it can be seen from FIGS. 3 and 4.

FIG. 3 shows the increase of gel-setting time as a function of the volume of added urea, in the case of constant final volume composition. The term “constant (standard) final volume composition” means that the combined volume of the methanol and the used additive in methanol was 11.0 ml, the volume of the silane reagent (e.g. tetramethoxysilane, TMOS) was 1.50 ml, and the volume of the water and the 1:1 diluted ammonia solution was 2.50, therefore the combined final volume of all components was 15 ml. The urea solution used in these studies, as well as the solutions of further potential additives used in later experiments were made by dissolving 2.00 g additive in 10.0 ml methanol (with mild heating if necessary). According to the figure, the gel-setting time at the given reaction mixture composition increases to 3000 s from the about 20 s measurable without the urea.

FIG. 4 shows the change of viscosity, characterized with fall time as a function of the volume of added urea, in the case of constant final volume composition. It can be seen that the gel-setting time can be varied within a broad range as a function of the amount of urea at any given composition.

### Example 2

### Variation of Gelation Time in Response to Different Additives

Based on the observations of Example 1, we carried out further experiments.

We measured in a series the effect of different substances on the gel-setting time, and obtained the approximate gel-setting time values shown in Table 1 below: (Conditions: 1.00 ml (or 1.00 g for solids) additive/5.00 ml MeOH stock solution. Solution “A”: 1.00 ml additive stock solution+10 ml MeOH, 1.50 ml TMOS; Solution “B”: 0.80 ml water+1.70 ml 1:1 diluted NH₃ solution.)

TABLE 1

| added substance | gelation time | |
|---|---|---|
| without additive | 18-19 | s |
| DMF | 260 | s |
| DMSO | 1130 | s |
| urea | 740 | s |
| ethylene glycol | 120 | s |
| propylene glycol | 25 | s |
| glycerol | 24 | s |
| ethanol | 18-19 | s (no gelation retarding effect) |
| 1-propanol | 18-19 | s (no gelation retarding effect) |
| 1-butanol | 18-19 | s (no gelation retarding effect) |

In another series, significant viscosity increasing effect was also observed at different concentrations. (Solution “A”: 6.0 ml of the additive examined+1.50 ml TMOS; Solution “B”: 5.0 ml methanol+1.50 ml water+0.75 ml 25% NH₃ solution). In the case of glycerol, the protocol was as follows: we used only 3.00 ml glycerol in Solution “A”, and less, about 0.50 ml 25% NH₃ in Solution “B”. Gelation times were measured only approximately, not as precisely as for the previous series. The results are summarized in Table 2 below.

TABLE 2

| added substance | gelation time | |
|---|---|---|
| without additive | 20-30 | s |
| propylene glycol | about 2 | hours |
| glycerol | about 1 | hour |
| fine cellulose powder | 15-20 | minutes |
| ethanol | 30 | s |

| | |
|---|---|
| i-propanol | a few seconds |
| n-butanol | a few seconds |

According to these experiments, therefore, propylene glycol, glycerol and the fine cellulose powder (this latter is a heterogeneous phase substance) have viscosity increasing and gel-setting time increasing effect.

We did not observe gelation retarding effect for the the monovalent alcohols in this series of experiments either, where fast gelation with opalescence occurred. Gelation happened for ethanol in 30 s, for i-propanol and n-butanol almost immediately (within a few seconds).

In summary: it was found that typically compounds that are capable of forming multiple hydrogen bonds (such as diols, polyols, dimethylformamide, dimethyl sulfoxide) are able to provide gelation retarding effect, while with simple alcohols that have only one OH group capable of forming such type of bonds, no advantageous results were achieved. Especially good results were obtained, in addition to urea, in the case of dimethylformamide, dimethyl sulfoxide, ethylene glycol, propylene glycol, glycerol and cellulose.

### Example 3

### Study of the Gelation Retarding Effect of DMSO

A more detailed study was performed with the additive DMSO, in which the changes were studied in function of the amount of catalyst. The results are shown in FIG. 5. The figure shows the apparent gel-setting time as a function of the volume of added DMSO additive in constant final volume compositions. Depending on the volume ratio of TMOS/concentrated NH₃, the same amount of DMSO shows a viscosity increasing effect with different characteristics. The solutions used:

Solution “A”: 50.0 ml MeOH+15.00 ml TMOS.

Solution “B”: 50.0 ml MeOH+17.00 ml 1:1 diluted 25% NH₃ solution+8.00 ml H₂O.

Additive solution: x ml DMSO+(1-x) ml MeOH.

The horizontal axis shows volume x of DMSO in the figure.

Compositions:

At the ratio of TMOS/conc. NH₃=2.35: 7.50 ml Solution “A”+6.50 ml Solution “B”+1.00 ml DMSO additive solution.

At the ratio of TMOS/conc. NH₃=1.76: 6.50 ml Solution “A”+7.50 ml Solution “B”+1.00 ml DMSO additive solution.

We note that in this figure the value of gel-setting time (“apparent gel-setting time”) is the time for the gel becoming so viscous that it is not capable to any further macroscopic movement. The values thus obtained are not the same as the results of the fall experiments carried out with the steel ball. The falling ball experiments cover a much wider viscosity range, since the high-density steel ball is still sinking in the gel, when it is not capable of spontaneous movement in the test tube used for the experiments.

### Example 4

### Simultaneous Catalyst and Gelation Retarding Effect with Diazatetraoxa-Crown Ether

Basic compounds capable of forming at least two hydrogen bonds also possess gelation retarding properties, and at the same time can play the role of the catalyst. We present experimental data on this in the following:

Solution “A”: 15 ml TMOS dissolved in 50 ml MeOH

Solution “B”: 1.00 ml water+200 ml crown ether solution

Crown ether solution: 292 mg 1,10-diaza-4,7,13,16-tetraoxa-cyclooctadecane dissolved in 3.00 ml water

Composition: 2.00 ml Solution “A” was admixed with Solution “B” under vigorous mixing. The reaction mixture heated up, then its viscosity increased continuously and it has gelled within 4 minutes 15 s, clear as glass.

### Example 5

### Studying the Gelation Retarding Effect of Pyridine

Solution “A”: 5.00 ml MeOH+1.00 ml pyridine+1.50 ml TMOS

Solution “B”: 50.0 ml MeOH+17.0 ml NH₃ solution (freshly prepared, 1:1 diluted)+8.00 ml water

Reaction mixture: 7.50 ml Solution A+5.00 ml Solution B, mixed in a strong jet

| Additive | Gelation time (s) |
|---|---|
| None (blank) | 64 |
| Pyridine | 84 (mean of four experiments) |

Based on this experiment it follows that pyridine also possesses viscosity increasing properties, although it is not very strong.

Silica Alcogel Composites Made with Urea Additive

### Example 6

### Alcogel Comprising Quartz Sand

Solution “A”: 6.0 ml methanol+1.50 ml TMOS; Solution “B”: 5.0 ml methanol, which contains dissolved 1.00 g urea, then 1.50 ml water and 1.00 ml 25% NH₃ solution. Solutions “A” and “B” were mixed in a test tube, then immediately 1.50 g washed quartz sand was added, then the tube was closed and the mixture was agitated and rotated by hand until the particles did not sink any more. Then the reaction mixture in the test tube was left stand until complete gelation. The gelation required the time of 2 hours.

### Example 7

### Alcogel Comprising Magnetite Microcrystals

The magnetite used (30-70 micron particle size, may have contained elemental iron particles for a certain degree) was made in-house with the reduction of Fe₂O₃ by carbon monoxide, at about 400° C. temperature. Quality control of the magnetite was carried out by a magnet, it did not contain non-magnetic particles.

Preparation of the alcogel was performed as described in Example 6, with the exception that 227 mg, in-house made magnetite was admixed. Gelation was completed in 30 minutes.

### Example 8

### Alcogel Containing 3 mm Glass Beads

Preparation of the alcogel was performed as described in Example 6, with the exception that we used 1.00 ml water and 1.50 ml 25% NH₃ solution in Solution “B”, and after mixing Solutions “A” and “B”, approximately 20 pieces of 3-4 mm glass beads were added. Gelation was completed in about 15 minutes.

### Example 9

### Alcogel Comprising Iron Powder

Preparation of the alcogel was performed as described in Example 6, with the exception that we used 1.00 ml water and 1.50 ml 25% NH₃ solution in Solution “B”, and after mixing Solutions “A” and “B”, we used 1.00 g iron powder. Gelation was completed in about 30 minutes.

### Example 10

### Alcogel Comprising Lead Sand

Preparation of the alcogel was performed as described in Example 6, with the exception that we used 1.00 ml water and 1.50 ml 25% NH₃ solution in Solution “B”, and after mixing Solutions “A” and “B”, we used 5.00 g lead sand. Gelation was completed in about 8 minutes.

### Example 11

### Alcogel Comprising Copper Powder

Preparation of the alcogel was performed as described in Example 6, with the exception that we used 1.00 ml water and 1.50 ml 25% NH₃ solution in Solution “B”, and after mixing Solutions “A” and “B”, we used 0.967 g copper powder. The reaction mixture gelled in about 5 minutes.

### Example 12

### Alcogel Comprising Iron(III)-Oxide

Preparation of the alcogel was performed as described in Example 6, with the exception that we used 1.00 ml water and 1.50 ml 25% NH₃ solution in Solution “B”. 528 mg Fe₂O₃ was suspended in Solution “A”, then the clumped parts were dispersed by immersing it into ultrasonic bath. After mixing Solutions “A” and “B”, the reaction mixture was treated with ultrasound several times intermittently in addition to agitation to avoid clumping. Gelation was completed in about 8 minutes.

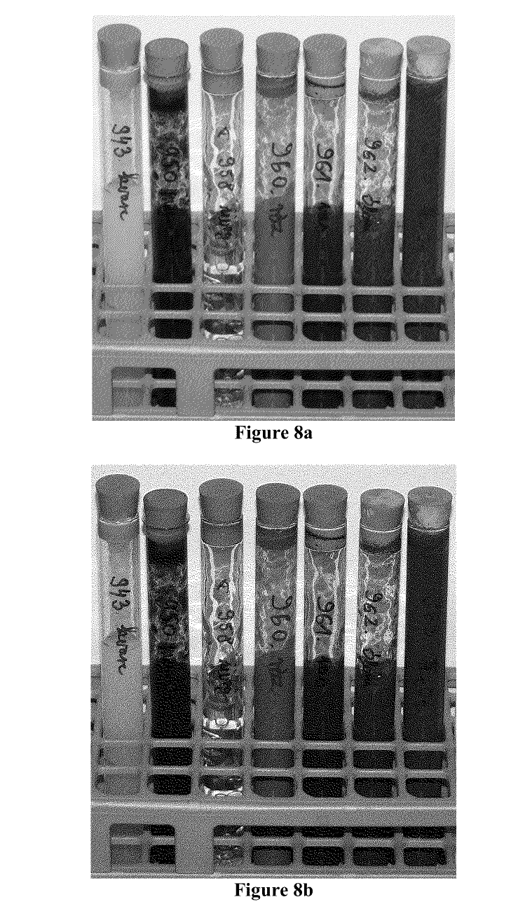

The photograph of the alcogels prepared in Examples 6 through 12 is visible in FIG. 8, from left to right in the following order: quartz sand, magnetite, glass beads, copper powder, iron powder, lead sand, iron(III)-oxide.

### Example 13

### Alcogel Composite Comprising Paraffin Oil and Aerogel Foam

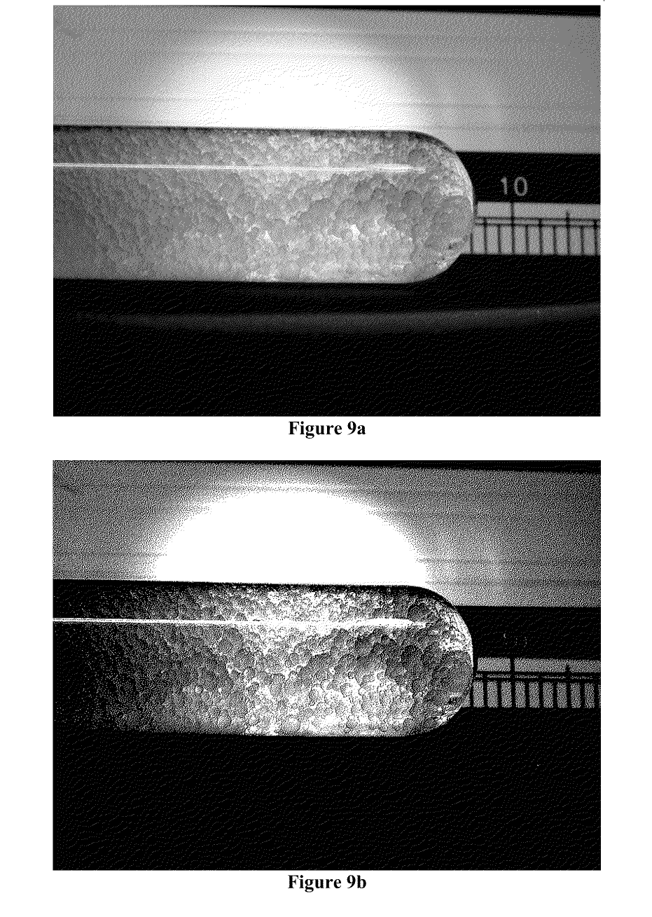

Two solutions were prepared: Solution “A”: 26.0 ml methanol, 4.00 ml freshly prepared urea solution in methanol (prepared by dissolving 10.0 g urea at about 50° C., under continuous stirring in 50.0 ml methanol, then after complete dissolution, the solution was cooled back to room temperature), 4.40 ml water, 5.60 ml NH₃ solution (prepared by dilution of 25% ammonia solution in 1:1 ratio). Solution “B”: 14.00 ml methanol, 6.00 ml TMOS. Solutions “A” and “B” were mixed with a magnetic stirrer, then 6.0 ml paraffin oil was mixed into it and the paraffin oil was dispersed by rotating and agitating with moderate intensity to avoid breaking up the droplets too much. The mixture was kept in continuous motion until it became thick, and then half the volume was poured into a mold, and the other half volume into a test tube, the samples were sealed air-tight and left to stand for a day. The photograph of the alcogel composite containing the dispersed paraffin oil is shown in FIG. 9.

After then, the gel within the mold was removed from the mold, and was subjected to gradual solvent replacement as described in Example 15. The remaining paraffin oil was removed from the gel with long term acetone wash. The aerogel was finally obtained from the alcogel by drying with supercritical carbon dioxide after an extraction with liquid carbon dioxide.

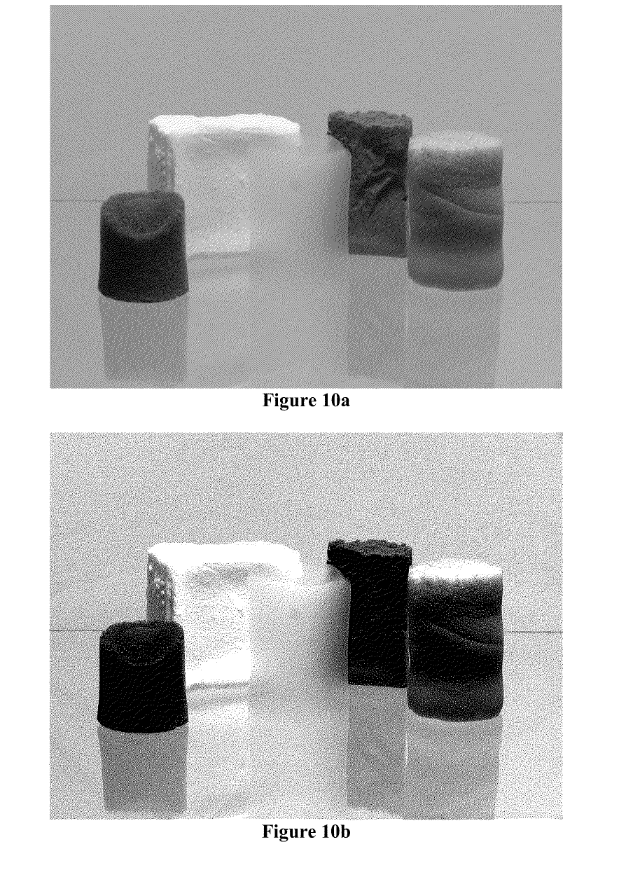

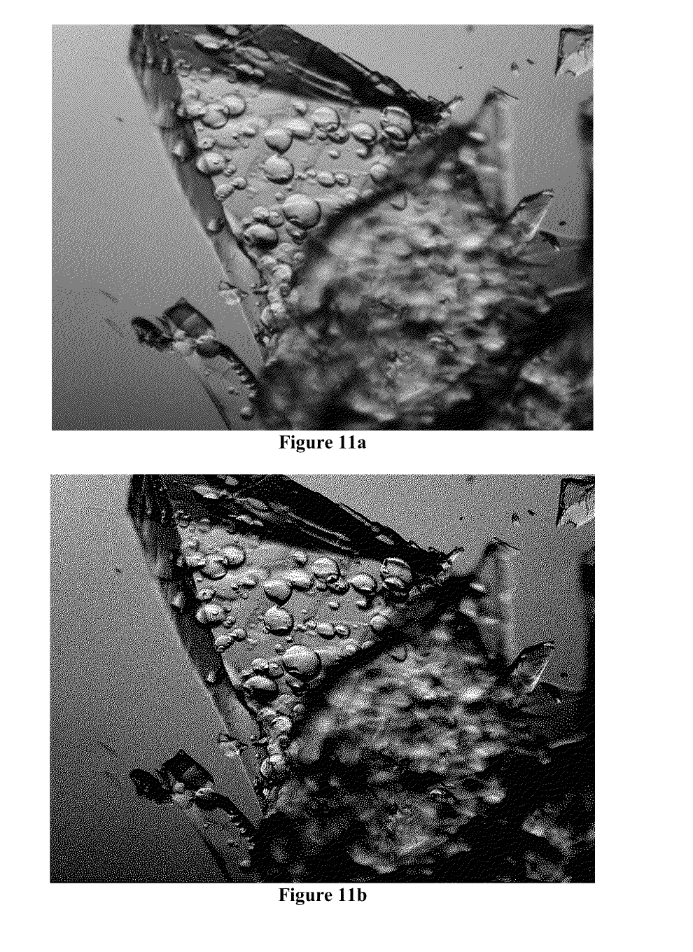

The picture of the aerogel foam containing air filled cavities in the place of paraffin droplets is shown in the middle of FIG. 10, and the microscopic picture of a part thereof is shown in FIG. 11. It is clear from FIG. 11 that after the dissolution of the oil droplets, gas (in the present case air) filled cavities remained. (The density of the oil droplets is 0.84 g/cm³, the density of the air in standard state is about 1.18·10⁻³ g/cm³).

### Example 14

### Alcogel Containing Polystyrene Foam Beads and Air Bubbles

Solution “A”: 3.50 ml MeOH+1.50 ml TMOS

Solution “B”: 6.50 ml MeOH+0.80 ml H₂O+1.70 ml 1:1 diluted NH₃ solution+1.00 ml urea additive solution.

Urea additive solution: 1.00 g urea dissolved in 5.00 ml methanol.

Solutions “A” and “B” were mixed with a magnetic stirrer, then poured into a test tube. Approximately 300 mg polystyrene foam beads were added (which were prepared by heat expansion of polystyrene beads; its density is about 0.3 g/cm³), the test tube was sealed and the thickening solution was agitated by shaking and rocking back and forth until air bubbles were mixed into between the polystyrene foam beads. The reaction mixture was kept in homogeneous dispersion by the rotation and agitation of the test tube until the composite alcogel matrix became self supporting. Cross-linking required about 3 minutes.

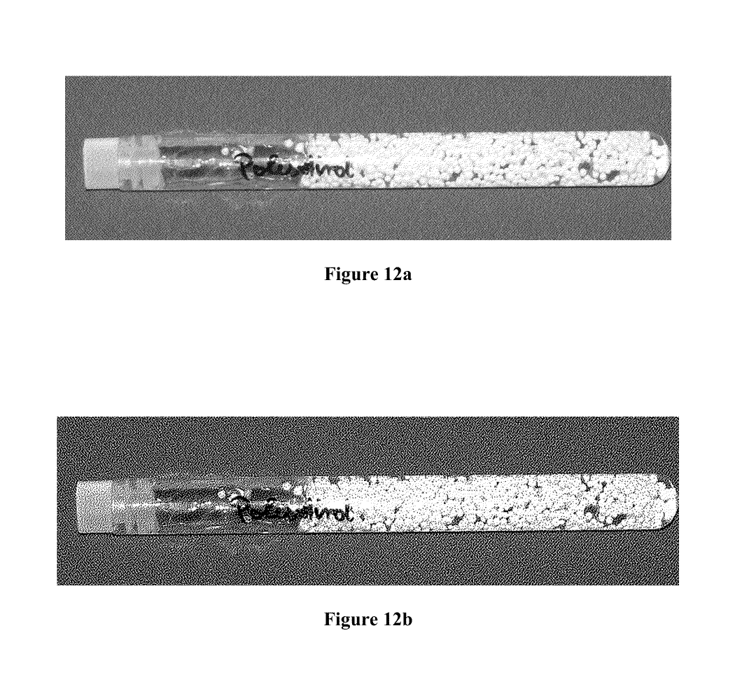

The picture of the alcogel obtained is shown in FIG. 12. It is clear that the low density (about 0.3 g/cm³) polystyrene foam beads are dispersed very evenly in the alcogel.

### Example 15

### Aerogel Composite Comprising Cr₂O₃

Solution “A”: 11.6 ml methanol+5.00 ml TMOS; Solution “B”: 15.0 ml methanol, 10.0 ml urea solution in methanol (prepared by dissolving 10.0 g urea in 50.0 ml warm methanol, then the solution was cooled down), 2.60 ml water, 5.00 ml 1:1 diluted NH₃ solution.

1.397 g anhydrous Cr₂O₃ was mixed to solution “B”, then the suspension was mixed with Solution “A” under vigorous stirring. The mixture obtained was intensively agitated by hand, stirred with magnetic stirrer, and then after reaching the desired viscosity, poured into a cylindrical mold that was sealed and left to stand for one day. After this, the alcogel obtained was forced out from the mold, and placed into a drying rack made of perforated aluminum sheet and subjected to gradual solvent exchange. First it was soaked in 100% methanol, then consecutively for one day each in 25%, 50%, 75% acetone-methanol mixtures, finally in acetone, which was exchanged several times. The gel with acetone was extracted in a tank reactor under pressure by streaming liquid carbon dioxide through it, finally was dried under supercritical conditions with carbon dioxide.

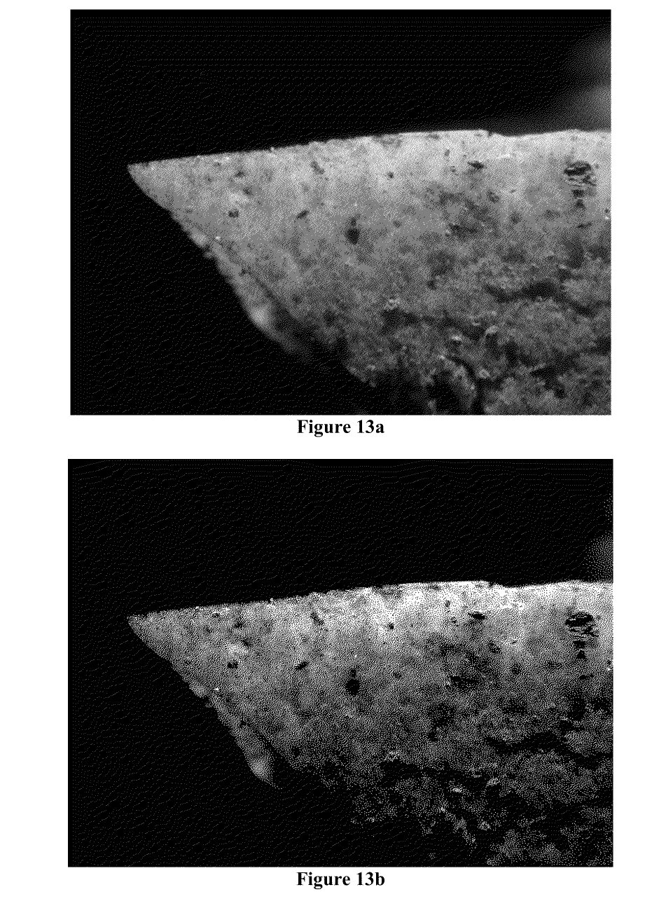

The picture of the aerogel composite obtained is shown in FIG. 10, and a microscopic picture of a fragment thereof is shown in FIG. 13. It is visible in FIG. 13 that small and large particles of the same substance may be dispersed simultaneously in the aerogel with this method (Density of Cr₂O₃: 5.22 g/cm³).

### Example 16

### Alcogel and Xerogel Composite with Calcium Phosphate and Cellulose

Solution “A”: 35.0 ml methanol+15.00 ml TMOS;

Solution “B”: 45.0 ml methanol, 30.0 ml urea solution in methanol, 8.00 ml water, 15.00 ml 1:1 diluted NH₃ solution.

Urea solution in methanol: 10.0 g urea is dissolved in 50.0 ml warm methanol, then the solution is cooled down to room temperature.

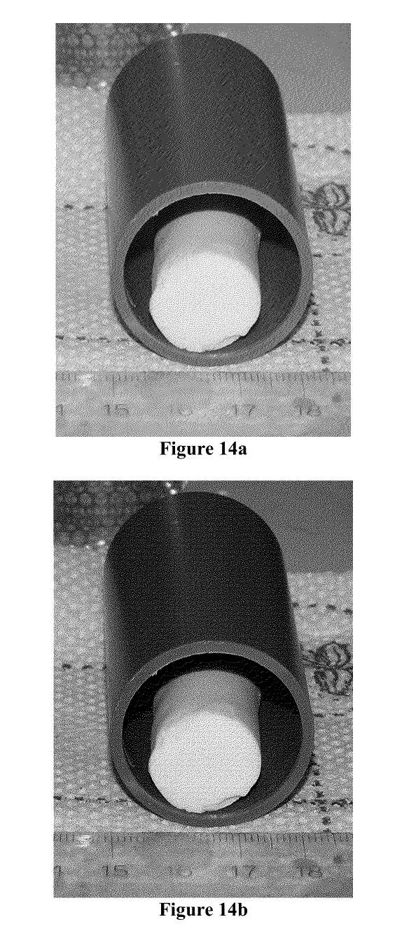

5.00 g calcium phosphate was added to Solution “B”, then mixed with Solution “A” under vigorous mixing. When the solid particles were dispersed evenly due to mixing, 5.00 g microcrystalline cellulose was added to the mixture. The reaction mixture was vigorously stirred, then agitated and shaked by hand until the desired thick, honey-like viscosity was achieved, then it was poured into a mold that was sealed and left to stand for one day. After this, the alcogel obtained was forced out from the mold, and placed into a cylinder made of five layers of filter paper and closed from each sides, and then left to dry slowly. During drying, the alcogel shrank evenly and significantly, without cracking. The composite xerogel obtained is shown in FIG. 14.

### Example 17

### Alcogel and Aerogel Comprising Lead Oxide

It was prepared by the method described in Example 10, with the exception that 4.168 g PbO was admixed to Solution “B”.

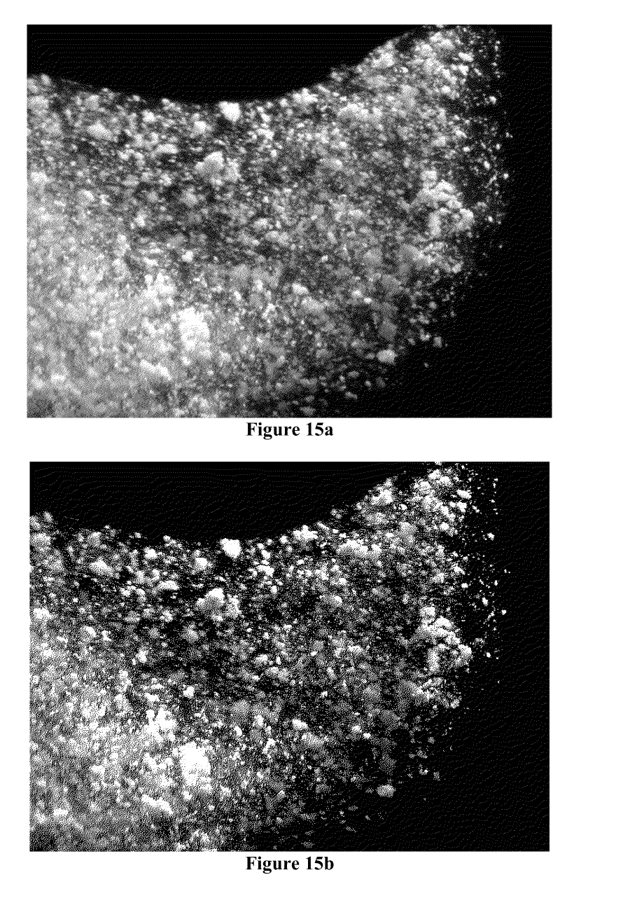

The picture of the aerogel composite obtained by the method is shown in FIG. 10 in the first row on the right, and a microscopic picture of a part thereof is shown in FIG. 15. It is clear from FIG. 15 that the high density (9.53 g/cm³) lead oxide has a very even distribution.

### Example 18

### Alcogel and Aerogel Comprising Calcium Phosphate, Hydroxyapatite and Cellulose

It was prepared by the method described in Example 15, with the exception that 0.83 g calcium phosphate was admixed to Solution “B”, then after mixing Solutions “A” and “B” together, 0.83 g hydroxyapatite and 1.60 g microcrystalline cellulose were added to the mixture, and after reaching the appropriate viscosity, the reaction mixture was poured into a rectangular mold.

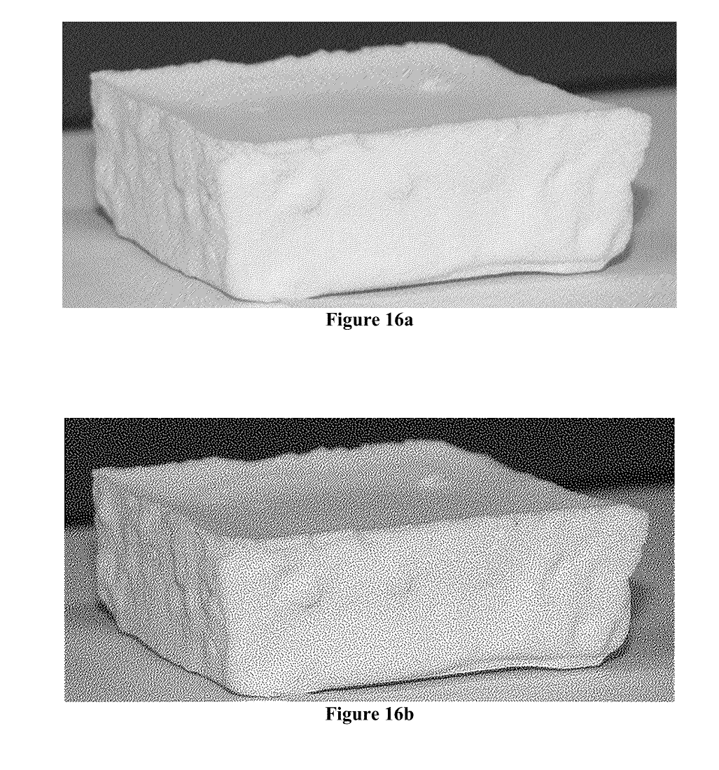

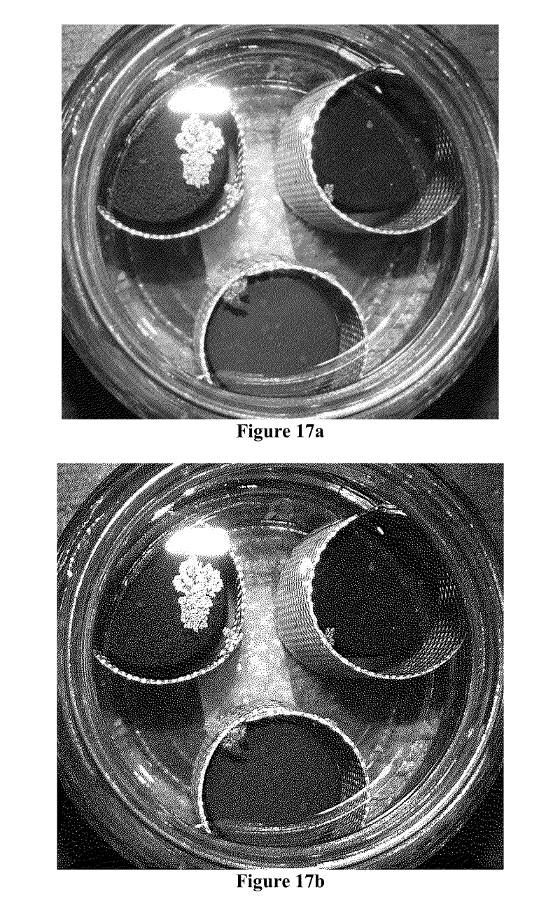

The picture of the aerogel obtained by the method is shown in FIG. 10 in the back on the left, as well as in FIG. 16.

Silica Alcogel Composites Made with DMF Additive

### Example 19

### Alcogel Comprising Lead Sand, and the Preparation of an Aerogel Therefrom

Two solutions were prepared, there were 18.00 ml DMF and 4.50 ml TMOS in Solution “A”, 15.00 ml MeOH, 4.50 ml H₂O and 3.00 ml 25% NH₃ solution in Solution “B”. 15.62 g lead sand (density: 11.34 g/cm³) was measured into an Erlenmeyer flask, to which Solutions “A” and “B” were added simultaneously under vigorous stirring. The suspension was initially stirred on a magnetic stirrer, then with the increase of the viscosity it was rotated and shaken by hand, and when the mixture became so viscous that the lead powder was evenly dispersed and did not sediment any more but was still plastic, then it was poured into a cylindrical mold that was sealed air-tight and it was cross-linked for one day. After this, the alcogel obtained was forced out from the mold, and placed into a drying rack and subjected to gradual solvent exchange. First it was soaked in 100% methanol, then consecutively in 25%, 50%, 75% acetone-methanol mixtures, finally in 100% acetone. The gel with acetone was extracted in tank reactor by liquid carbon dioxide, and finally was dried under supercritical conditions with carbon dioxide.

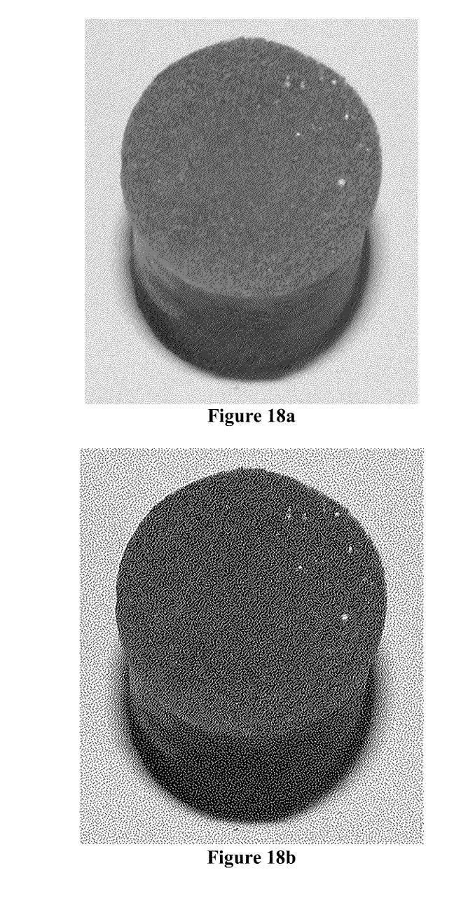

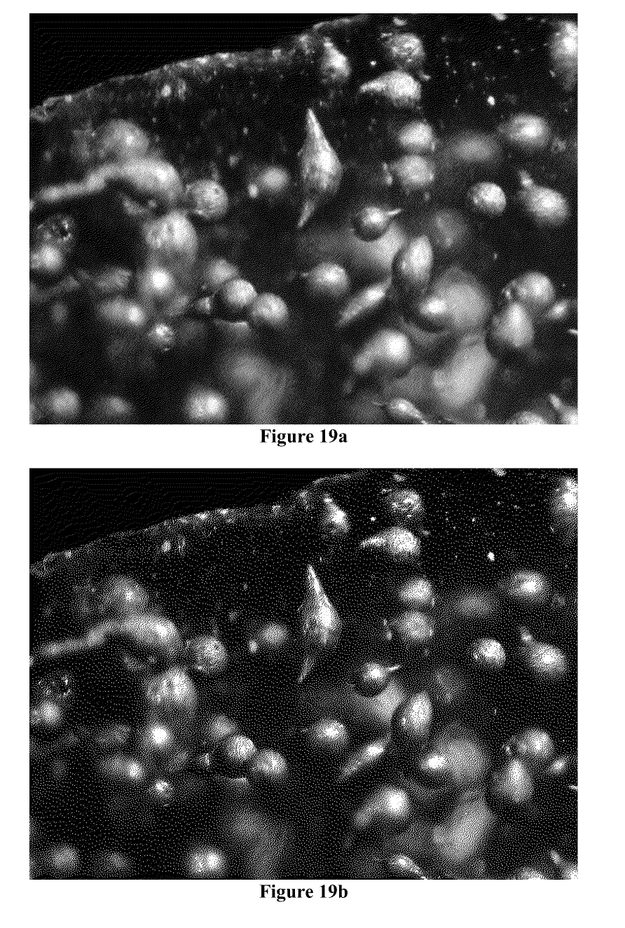

The alcogel containing lead sand is shown in FIG. 17 top left, the aerogel obtained therefrom is shown in FIG. 10 in the front row on the left side and in FIG. 18. The microscopic picture of the aerogel composite (magnification: 125×) is shown in FIG. 19. It is clear from the figures that the distribution of the high density (11.34 g/cm³) lead sand is uniform both in the alcogel and the aerogel.

### Example 20

### Alcogel and Aerogel Comprising Cu Powder

It was prepared according to the method described in Example 17 from the following materials. Solution “A”: 18.0 ml DMF, 4.50 ml TMOS, 1.762 g Cu powder; Solution “B”: 15.0 ml methanol, 4.50 ml water, 3.00 ml 25% NH₃ solution. The two solutions/suspensions were mixed under vigorous stirring, then after the beginning of thickening, it was poured into a mold, sealed air-tight and left to stand for one day. Then it was extracted according to the method described and dried with supercritical CO₂.

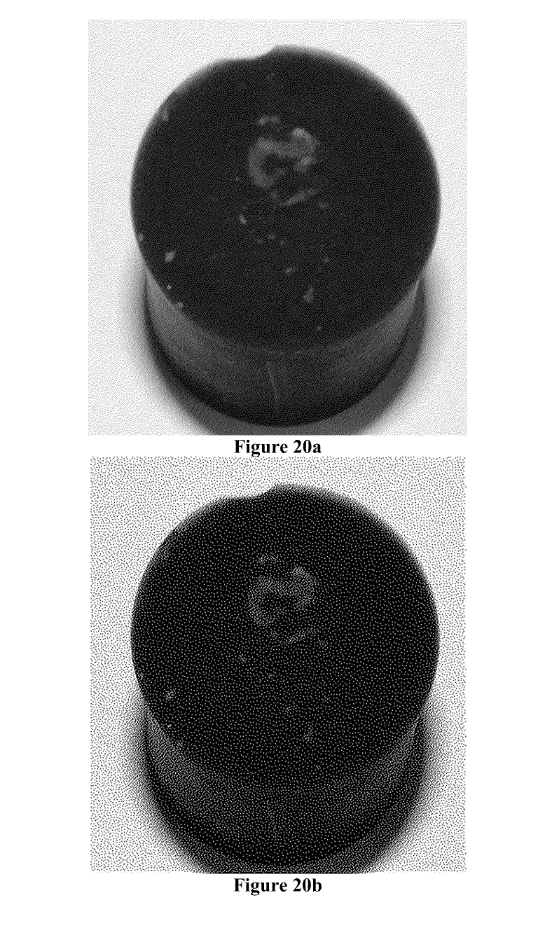

The alcogel and aerogel composites in the drying racks are shown in FIG. 17 in the bottom row, and the picture of the prepared aerogel composite tinted blue from the leached copper ions is shown in FIG. 20. It is clear from the figures that the distribution of the high density (8.96 g/cm³) copper powder is uniform both in the alcogel and the aerogel.

### Example 21

### Alcogel and Aerogel Comprising Fe Powder

It was prepared according to the method used for the gel comprising lead powder (Example 18) from the following materials. Solution “A”: 18.0 ml DMF, 4.50 ml TMOS, 10.19 g iron powder; Solution “B”: 15.0 ml methanol, 4.50 ml water, 3.00 ml 25% NH₃ solution. The two solutions/suspensions were mixed under vigorous stirring, then after the beginning of thickening, it was poured into a mold, sealed air-tight and left to stand for one day. Then it was extracted according to the method described in Example 15 and dried with supercritical CO₂.

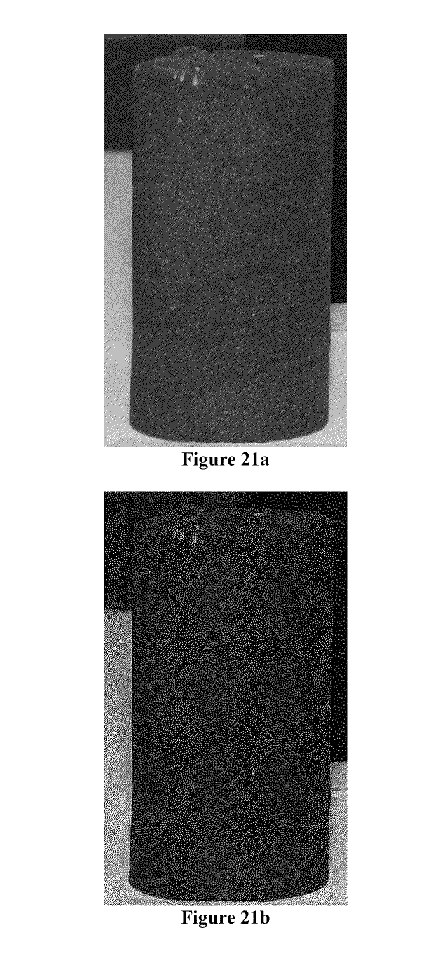

The picture of the alcogel comprising iron powder is shown in FIG. 17 top right, the picture of the aerogel is shown in FIG. 21. It is clear from the figures that the distribution of the iron powder is uniform both in the alcogel and in the aerogel.

### Example 22

### Alcogel and Aerogel Comprising Large Glass Beads

It was prepared according to the method used for the gel comprising lead powder (Example 18) from the following materials. Solution “A”: 36.0 ml DMF, 9.00 ml TMOS, 3-4 mm diameter glass beads of an apparent volume of 14 ml; Solution “B”: 30.0 ml methanol, 9.00 ml water, 6.00 ml 25% NH₃ solution. Under vigorous stirring, the two solutions were mixed together, the glass beads were added immediately, then it was shaken, agitated by hand until the viscosity increased very significantly. When the beads did not sediment any more but the material was still plastic, it was poured into a rectangular mold made of glass and silicone rubber, sealed air tight and left stand for one day. Then the solvent was exchanged according to the method described in Example 15, then it was extracted with carbon dioxide and dried with supercritical CO₂.

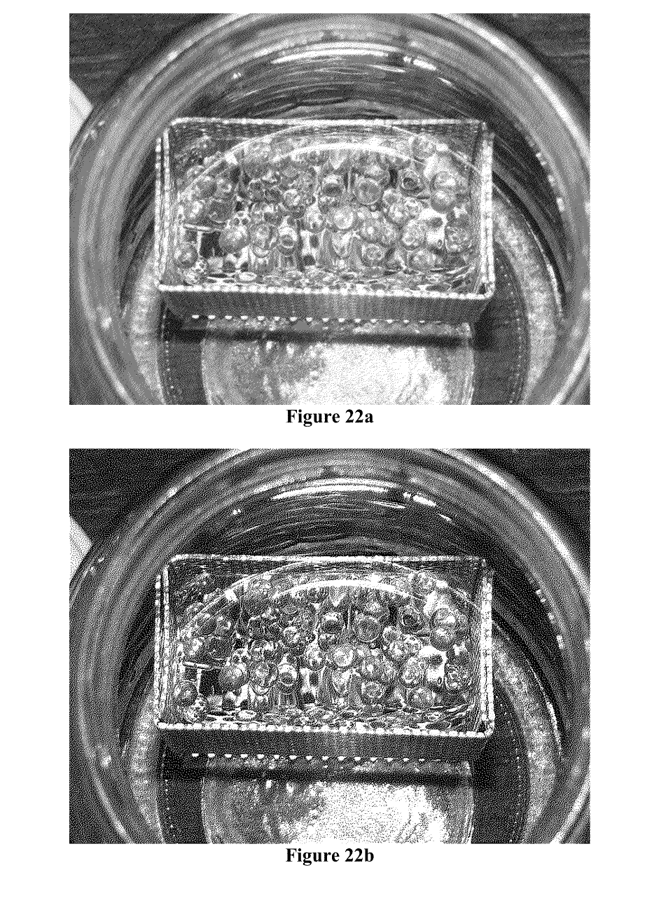

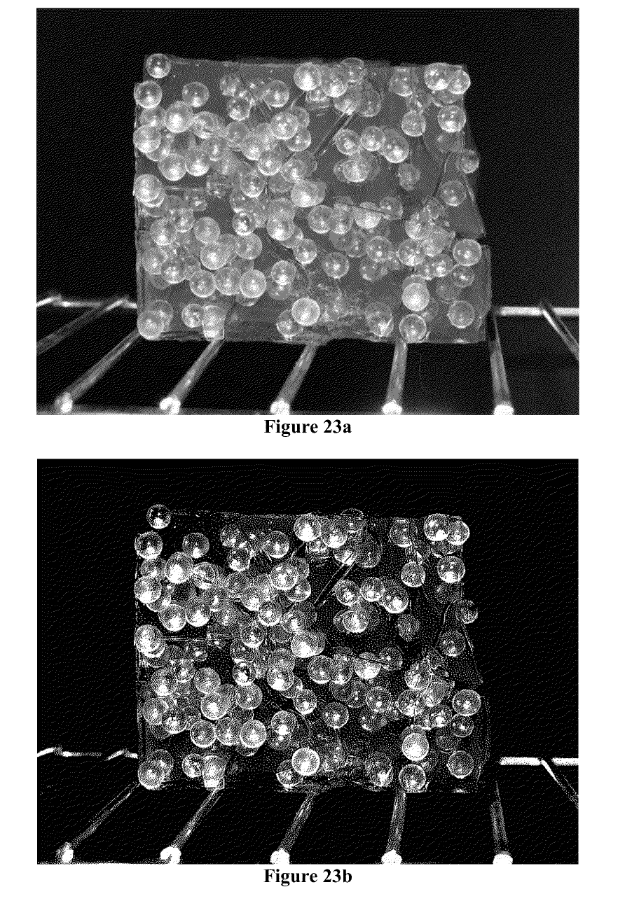

The alcogel obtained is shown in the drying rack in FIG. 22, and the picture of the prepared aerogel composite after drying is shown in FIG. 23. It is clearly seen on the figures that the large glass beads (3-4 mm in diameter), having a density of 2.5 g/cm³ are distributed evenly in the matrix.

### Example 23

### Alcogel, Aerogel and Macroporous Aerogel Comprising Cellulose

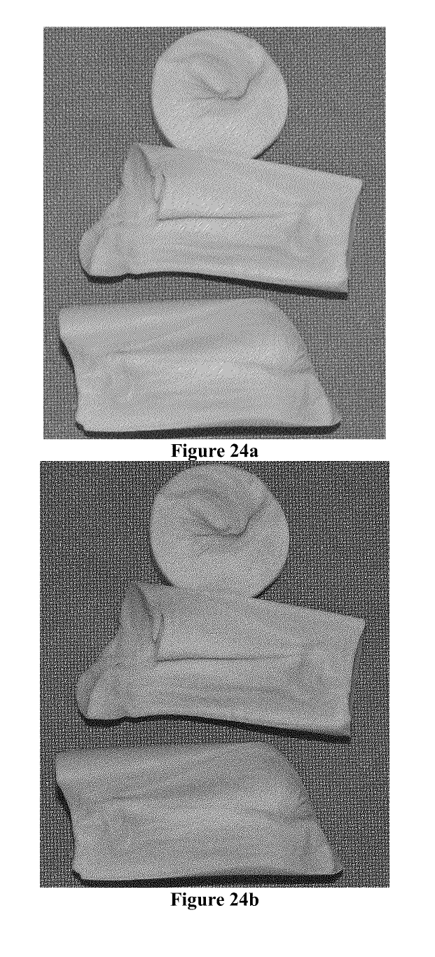

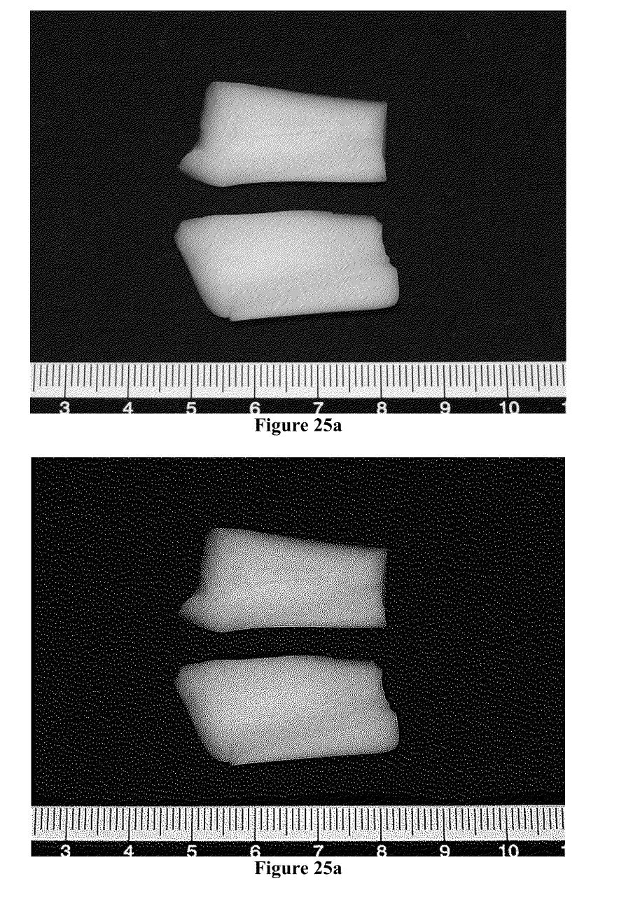

It was prepared according to the method used for the gel comprising lead powder (Example 18) from the following materials. Solution “A”: 18.0 ml DMF, 4.50 ml TMOS, 0.9943 g micro-crystalline cellulose; Solution “B”: 15.0 ml methanol, 4.50 ml water, 3.00 ml 25% NH₃ solution. The two solutions/suspensions were mixed under vigorous stirring, then after the beginning of thickening, it was poured into a mold, sealed air-tight and left to stand for one day. Then the solvent was exchanged according to the method described in Example 15, extracted, and dried with supercritical CO₂. The picture of the aerogel composite obtained after supercritical drying is shown in FIG. 24, the picture of the macroporous aerogel obtained after drying at 500° C. for 8 hours is shown in FIG. 25. It can be observed on the figures that after calcination, the white aerogel became opalescent, therefore a macroporous, but light-permeable aerogel can be prepared by the heat treatment, an on the other hand, the aerogel did not fall apart or disintegrated during calcination but kept its shape.

Silica Alcogel Composites Made with DMSO Additive

### Example 24

### Alcogel Containing Substances with Different Density and Different State of Matter (Polystyrene Beads and Air Bubbles) at the Same Time

Solution “A”: 5.00 ml MeOH+1.50 ml TMOS;

Solution “B”: 5.40 ml MeOH+0.80 ml H₂O+1.70 ml 1:1 diluted NH₃ solution+0.60 ml DMSO.

Solutions “A” and “B” were mixed with a magnetic stirrer, then poured into a test tube. 300 mg polystyrene beads were added, the test tube was closed and the thickening solution was agitated by intensively shaking until the desired amount of air bubbles were mixed in-between the polystyrene beads. The reaction mixture was kept in homogeneous dispersion by the rotation and agitation of the test tube until the composite alcogel matrix became self-supporting. Cross-linking required about 3-4 minutes.

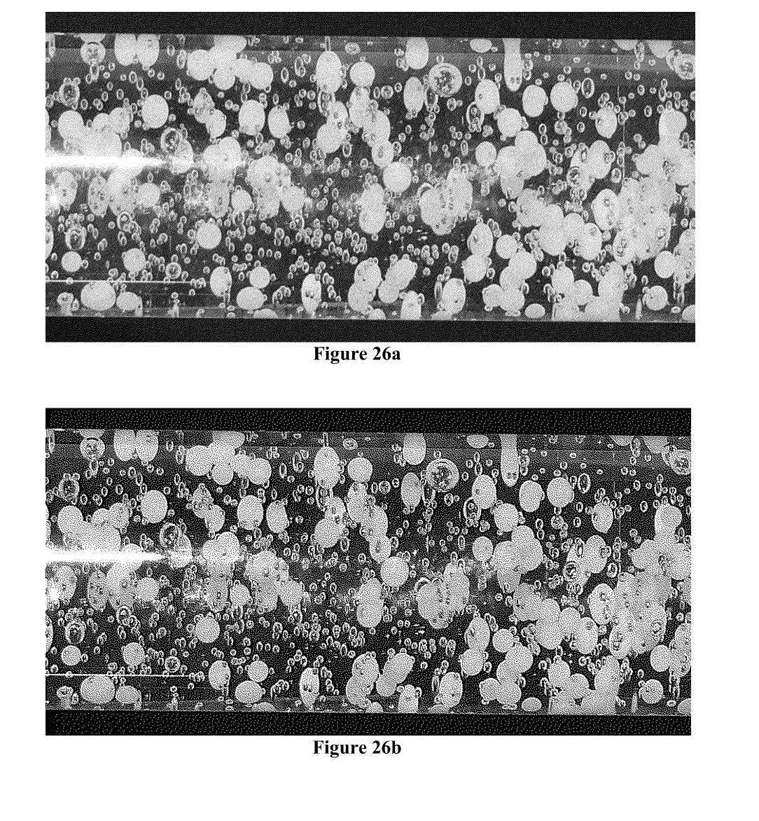

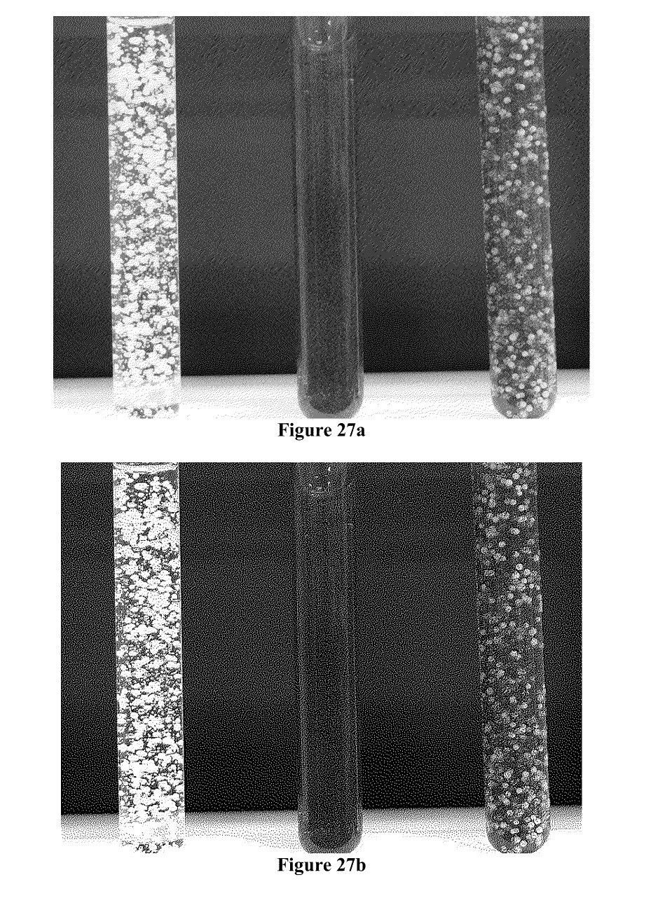

The picture of a part of the alcogel composite obtained is shown enlarged in FIG. 26, and the original size is shown on the left side of FIG. 27. The polystyrene beads are opaque on the figure, the air bubbles are shiny transparent. The uniform distribution of the particles with different state of matter and density (air: about 1.2*10⁻³ g/cm³, polystyrene bead: 1.18 g/cm³) is clearly visible.

### Example 25

### Alcogel Composite Comprising Lead Sand

It was prepared by the method described in Example 24, with the exception that 3.00 g lead sand was added instead of the polystyrene beads, and the reaction mixture was not shaken intensively, only as was necessary to obtain the uniform distribution of the particles. The picture of the alcogel composite obtained is shown on the middle of FIG. 27.

### Example 26

### Alcogel Composite Containing Solid Substances with Different Densities (Lead Sand and Polystyrene Beads) at the Same Time

It was prepared by the method described in Example 24, with the exception that 300 mg polystyrene beads, then 3.00 g lead sand were added, and the reaction mixture was not shaken intensively, only as was necessary to obtain the uniform distribution of the particles.

The picture of the alcogel composite obtained is shown on the right side of FIG. 27. The uniform distribution of the particles with different densities (lead: 11.3 g/cm³, polystyrene beads: 1.18 g/cm³) is clearly visible.

### Example 27

### Alcogel Composite Containing Solid Substances with Different Densities Lead and Polystyrene Foam Beads) at the Same Time

Solution “A”: 100 ml MeOH+30 ml TMOS;

Solution “B”: 100 ml MeOH+34 ml 1:1 diluted 25% NH₃ solution+16 ml H₂O.

Composition: a mixture of 7.50 ml Solution “B”, 0.90 ml DMSO and 0.10 ml MeOH was added to 6.50 ml Solution “A” under magnetic stirring, then the reaction mixture was immediately filled into a test tube containing 300 mg polystyrene foam beads. The mixture in the closed test tube was carefully agitated and shaken until it became viscous as honey and the beads dispersed uniformly within. Then the test tube was opened and 3.30 g lead sand was added thereto. After closing the tube again, the agitation and shaking was continued until complete gelation. Setting of the gel required about 6 minutes.

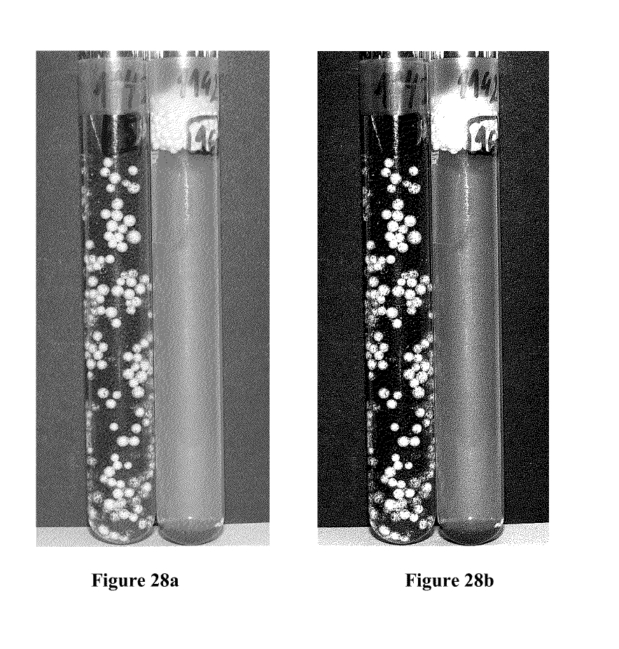

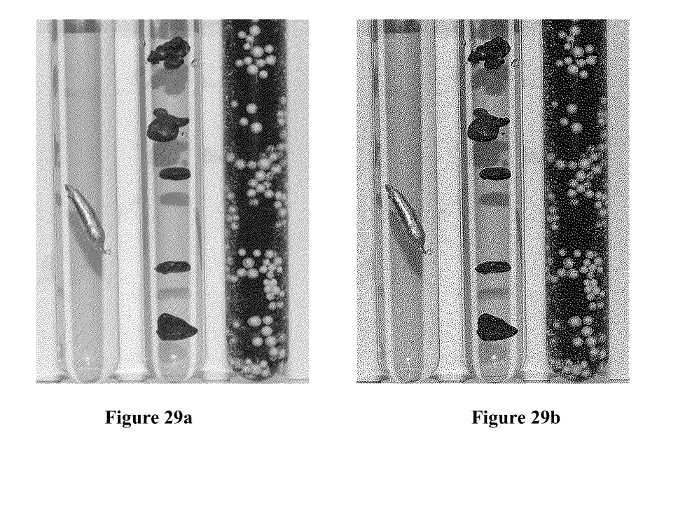

The picture of the alcogel composites obtained is shown on the left side of FIG. 28, and in FIG. 29. The uniform distribution of the particles with different densities (lead: 11.3 g/cm³, polystyrene foam beads: 0.3 g/cm³) is clearly visible.

### Example 28

### Alcogel Composite Containing a Piece of Tin

It was prepared according to the example described in Example 27, with the exception that after mixing the Solutions “A” and “B”, the mixture was filled into an empty test tube, and a single piece of tin of about 20 mm in length, 4 mm thick was dropped therein, the test tube was closed, then with careful agitation, tilting of the tube, the tin piece was positioned at the middle of the test tube in the central axis, where it was fixed during cross-linking without touching the wall.

The picture of the silica alcogel composite thus obtained is shown on the left side of FIG. 29. The density of tin: 7.3 g/cm³. This example clearly shows that due to the progressive nature of gelation, very large and high density material can be fixed within the gel without sinking.

### Example 29

### Silica Alcogel Composite Containing Lead Lumps

It was prepared according to the example described in Example 23, with the exception that after mixing the Solutions “A” and “B”, the mixture was filled into an empty test tube, and five lead lumps were dropped therein, the test tube was closed, then with careful agitation, tilting of the tube, the lead lumps were uniformly distributed at the central axis of the test tube, where they were fixed during cross-linking without touching the wall.

The picture of the silica alcogel composite thus obtained is shown on the middle of FIG. 29. This example clearly shows that due to the progressive nature of gelation, several blocks of very large and high density material (the density of lead is 11.4 g/cm³) can be fixed within the gel evenly.

### Example 30

### Silica Alcogel Composite Containing Polypropylene Particles

Solution “A”: 5.00 ml MeOH+1.50 ml TMOS;

Solution “B”: 5.10 ml MeOH+0.80 ml H₂O+1.70 ml 1:1 diluted NH₃ solution+0.90 ml DMSO.

Solutions “A” and “B” were mixed with a magnetic stirrer, then poured into a test tube. 5 g polypropylene beads were added, the test tube was closed, and the thickening solution was agitated until the polypropylene beads dispersed evenly, then the reaction mixture was kept in homogeneous dispersion by the rotation and agitation of the test tube until the composite alcogel matrix became self-supporting. Cross-linking required about 3-4 minutes.

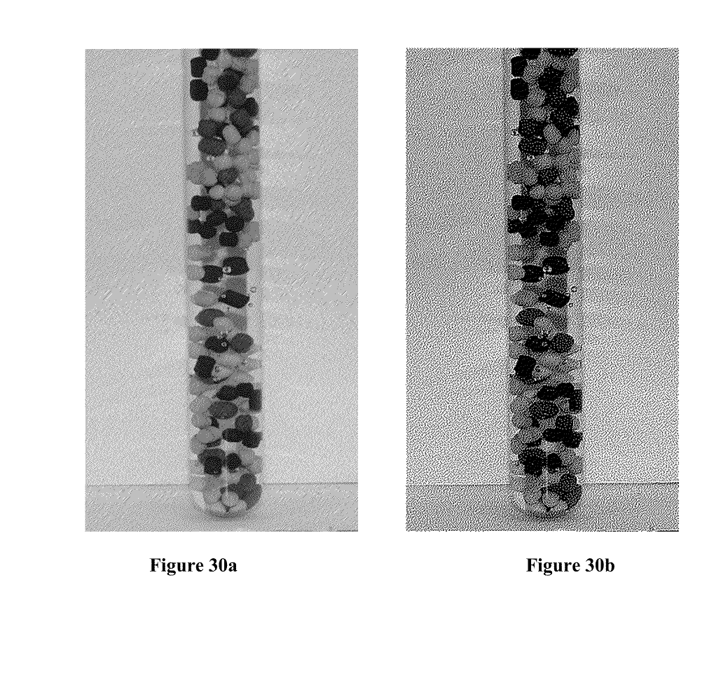

The picture of the alcogel composite is shown in FIG. 30.

### Example 31

### Silica Alcogel Composite Containing Nitrogen Bubbles

Solution “A”: 5.00 ml MeOH+1.50 ml TMOS

Solution “B”: 6.30 ml MeOH+0.80 ml H₂O+1.70 ml 1:1 diluted NH₃ solution+0.70 ml DMSO.

Solutions “A” and “B” were mixed by mixing with a magnetic stirrer, then we waited until the mixture had a viscosity of an oil. Then it was filled into a vertically positioned glass tube provided with a porous glass plate at the bottom, and through this plate, nitrogen was streamed into the mixture under pressure, thereby forming bubbles. The bubbles were further dispersed with the help of a hand-rotated, ribbed mixer body. The rate of feeding of the gas was decreased in parallel with the increase of viscosity, then stopped, and the mixing was maintained until the significant increase of viscosity. The cross-linking of the mixture occurred in about 4 minutes.

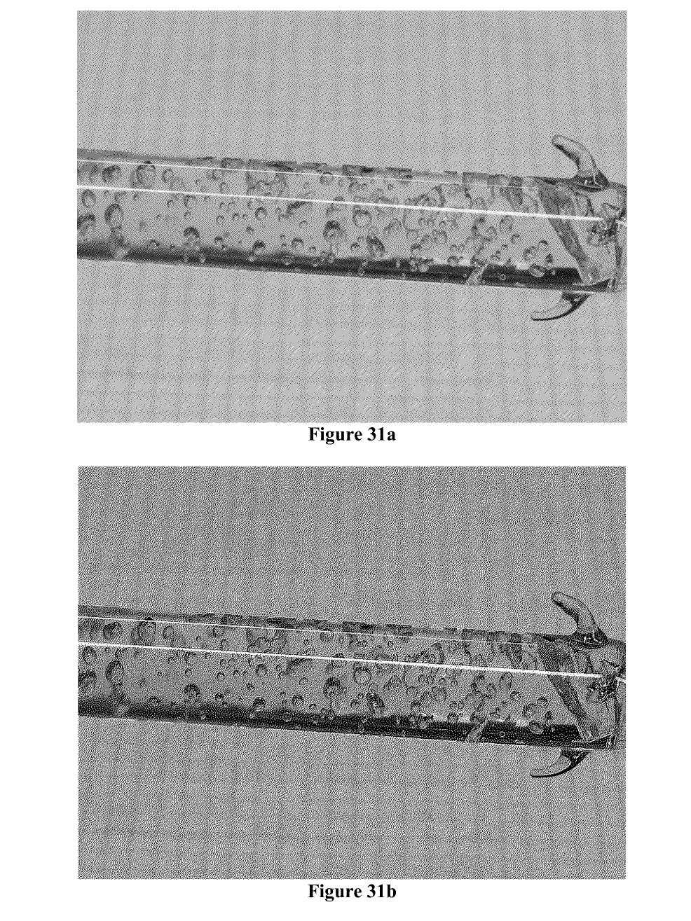

The picture of the alcogel composite thus obtained is shown in FIG. 31. It can be observed that the distribution of the nitrogen bubbles (density: 1.25 g/l) is uniform, they do not emerge to the top of the alcogel.

Silica Alcogel Composite Made without Additive (Comparative Example)

### Example 32

### Silica Alcogel, Comprising Lead Sand and Polystyrene Foam Beads, Made without the Addition of an Additive (Comparative Example)

Solution “A”: 100 ml MeOH+30 ml TMOS;

Solution “B”: 100 ml MeOH+34 ml 1:1 diluted 25% NH₃ solution+16 ml H₂O.

Composition: 8.50 ml Solution “B” was added to 6.50 ml Solution “A”, then the reaction mixture was immediately filled into a test tube containing 300 mg polystyrene foam beads, and 3.3 g lead sand was quickly added, and the test tube was sealed and its content was mixed by turning over twice. The added lead sand and the polystyrene foam beads immediately sunk down and emerged to the surface, respectively. There was no chance for turning over a third time, since the cross-linking completed in 21 s.

FIG. 28 on the right side shows a picture of an alcogel comprising the sorted particles, made without an additive.

FIG. 28 clearly shows that dispersion of heterogeneous particles cannot be achieved without additive, rather these emerge or sink depending on their densities upon addition to the reaction mixture in the available short time, and the is gel cross-linked within a few seconds (right hand side test tube). With an additive, the appropriately uniform dispersion is possible even with particles having very different densities (left hand side test tube; prepared according to Example 27).

Silica aerogel composite and macroporous aerogel made with cellulose additive (the cellulose is a gelation retarding additive, dispersed particle and calcifiable pore-forming agent at the same time)

### Example 33

### Aerogel and Macroporous Aerogel Containing Microcrystalline Cellulose

Solution “A”: 50.0 ml methanol, 12.6 ml TMOS;

Solution “B”: 42.0 ml methanol, 8.40 ml water and 10.00 ml 25% NH₃ solution.

In a beaker, 2.108 g chromatography-grade microcrystalline cellulose was added to Solution “A”, then the Solution “B” was added under magnetic stirring. The beaker was shaken by hand and rotated, mixed moderately with a spatula as necessary. The agitation was continued until the mixture had a honey-like viscosity, and the particles did not sediment any more. Then the reaction mixture was poured into molds and left to stand after sealing. Gelation required about 30 minutes.

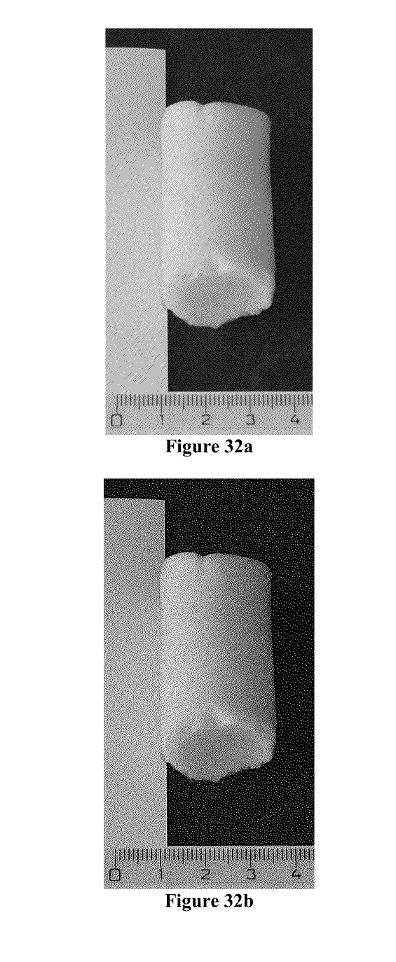

The alcogel thus formed was removed from the mold after one day and was dried with supercritical carbon dioxide after solvent exchange and extraction as described in Example 15. The picture of the aerogel obtained after drying is shown in FIG. 32.

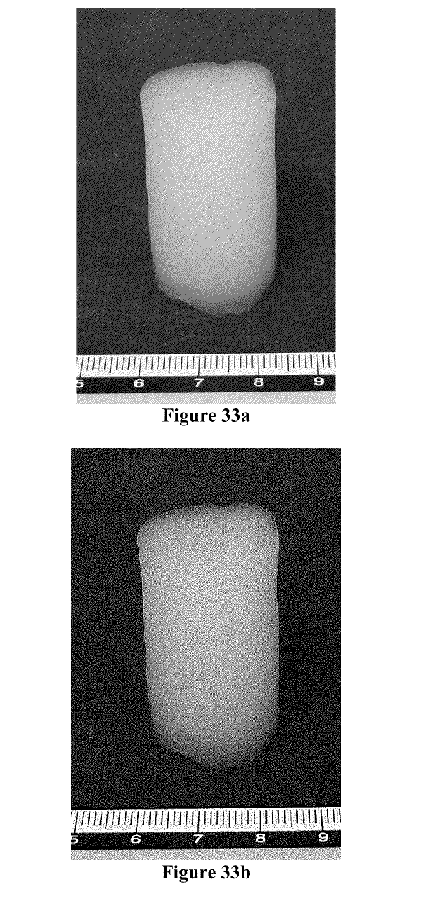

The obtained aerogel was heated at 600° C. temperature for 3 hours in an oven. The cellulose particles dispersed in the aerogel burned out during this time, and pores remained in their place. The picture of the macroporous aerogel thus obtained is shown in FIG. 33.

Composites Made with Compounds Providing Catalyst and Gelation Retarding Effect at the Same Time

### Example 34

### Simultaneous Catalyst and Gelation Retarding Effect with Diethylenetriamine, Composite with Polypropylene Beads

Solution “A”: 15 ml TMOS dissolved in 50 ml MeOH;

Solution “B”: 2.00 ml water+0.05 ml diethylenetriamine.

Composition: 3 mm diameter polypropylene beads were added to 3.00 ml Solution “A”, then Solution “B” was added under intensive agitation. The reaction mixture warmed slowly, then its viscosity gradually increased, the mixture was agitated until gelation to ensure the uniform dispersion of the beads. The complete gelation required about 40 minutes.

### Example 35

### Simultaneous Catalyst and Gelation Retarding Effect with Tetramethylethylene-diamine, Composite with Polypropylene Beads

Solution “A”: 15 ml TMOS dissolved in 50 ml MeOH;

Solution “B”: 2.00 ml water+0.05 ml tetramethylethylenediamine.

Composition: 3 mm diameter polypropylene beads were added to 3.00 ml Solution “A”, then Solution “B” was added under intensive stirring. The reaction mixture warmed, its viscosity increased. The mixture was appropriately agitated by hand until complete gelation to ensure the uniform dispersion of the particles. The complete gelation required about 3 minutes.

### Example 36

### Simultaneous Catalyst and Gelation Retarding Effect with Piperazine, Composite with Polypropylene Beads

Solution “A”: 15 ml TMOS dissolved in 50 ml MeOH;

Solution “B”: 2.00 ml water+0.20 ml piperazine solution;

Piperazine solution: 660 mg piperazine dissolved in 6.00 ml water.

Composition: 3 mm diameter polypropylene beads were added to 3.00 ml Solution “A”, then Solution “B” was added under intensive shaking. The reaction mixture warmed, then its viscosity gradually increased. The mixture was appropriately agitated by hand until gelation to ensure the uniform dispersion of the particles. The complete gelation required 5 minutes.

### Example 37

### Simultaneous Catalyst and Gelation Retarding Effect with 2,2′-(ethylenedioxy)-diethylamine, Composite with Polypropylene Beads

Solution “A”: 15 ml TMOS dissolved in 50 ml MeOH;

Solution “B”: 2.00 ml water+0.10 ml 2,2′-(ethylenedioxy)diethylamine solution;

2,2′-(ethylenedioxy)diethylamine solution: 0.60 ml 2,2′-(ethylenedioxy)diethyl-amine dissolved in 5.00 ml water.

Composition: 3 mm diameter polypropylene beads were added to 3.00 ml Solution “A”, then Solution “B” was added under intensive shaking. The reaction mixture warmed, then its viscosity gradually increased. The mixture was appropriately agitated by hand until complete gelation to ensure the dispersion of the particles. The complete gelation required 5 minutes.

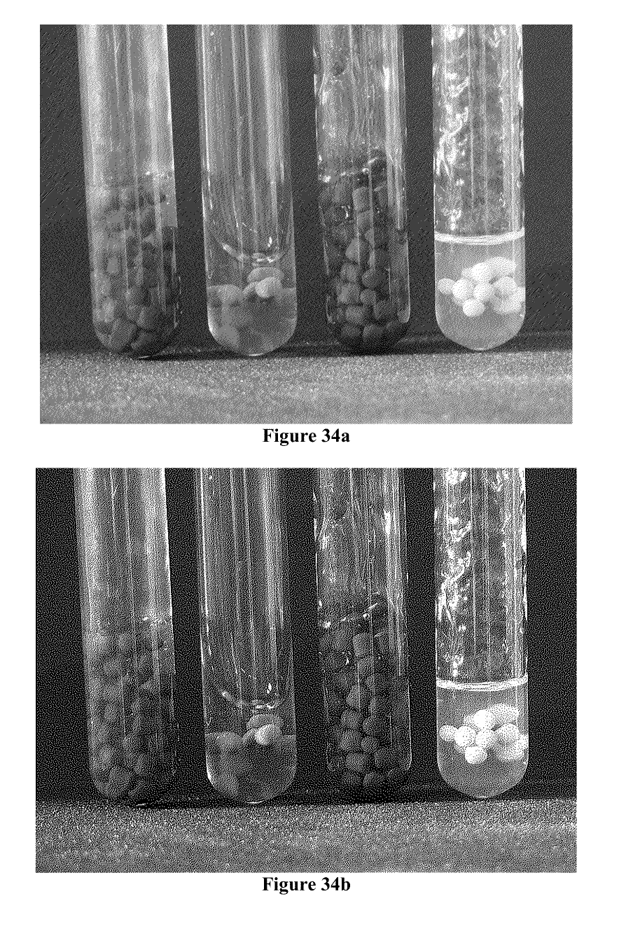

The photographs of the alcogel composites prepared according to Examples 34-37 are shown in FIG. 34, the alcogel composites obtained in Example 34, 35, 36 and 37 are shown from left to right.

Example for the Application of Continuous Manufacturing Technology

### Example 38

### Preparation of an Alcogel Composite Comprising Hydroxyapatite with Continuous Technology

Solution “A”: mixture of 70 ml methanol and 30 ml TMOS;

Solution “B”: 66 ml methanol, 23 ml H₂O, 10.00 ml 25% ammonia solution, 5.00 g powdered urea.

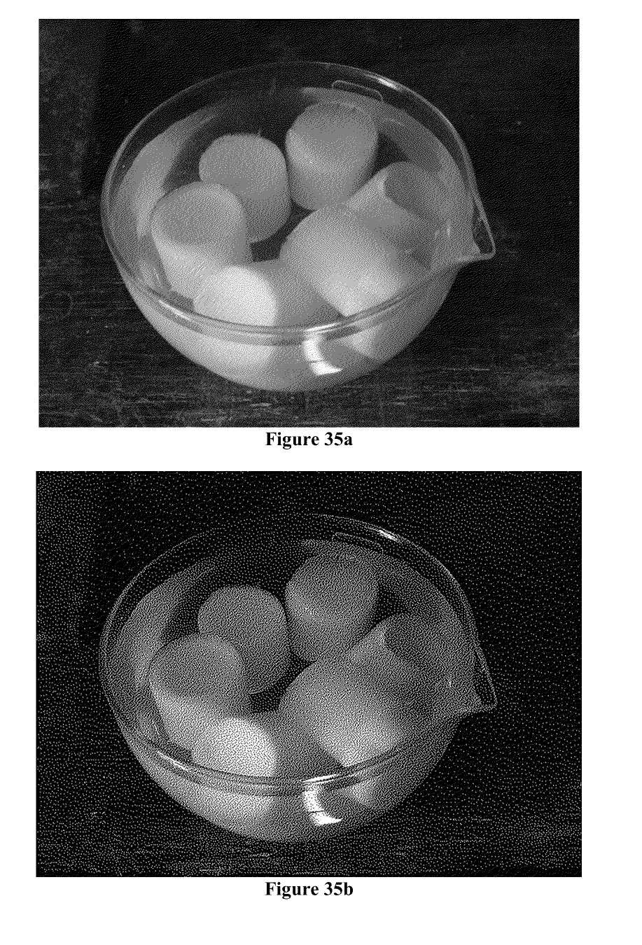

The separately prepared Solutions “A” and “B” were measured into dropping funnels, and 20 g powdery hydroxyapatite was measured into a screw feed funnel suitable for the addition of powders. All the mixers in the reactor and the rotation of the tubular reactor were started. The feeding of the two reagents were set to a rate of 2 drops/s, then at the same time the uniform feed of the hydroxyapatite was initiated. Solutions “A” and “B”, as well as the hydroxyapatite were fed simultaneously and in a synchronized manner into a vertically positioned primary mixing chamber provided with a worm mill and rotating mixer elements, where the reaction mixture was formed and homogenized, which was transferred by an Archimedean screw type mixer through a nearly horizontal secondary mixing chamber to the rotating tubular reactor that was initially tilted at 5°, then after the first 3 minutes, at about 10°. The gradually thickening reaction mixture progressed through the tubular reactor on a helical path, while rubber rollers contacting the wall ensured at the top point of the reactor tube the separation of the reaction mixture from the wall and the further gentle mixing thereof. The reaction mixture achieving the appropriate viscosity fell into cylindrical plastic molds after reaching the bottom point of the tubular reactor. During the process, five pieces of complete samples with about 30-35 ml volume each were obtained, as well as one sample that only partially contained particles (after running out of hydroxyapatite). The samples completely gelled within 15-20 minutes after filling the mold. The alcogels thus obtained are shown in FIG. 35.

As it is apparent from the description and the specific examples, the advantage of the invention is that it provides for the uniform dispersion of guest particles with arbitrary state of matter and density, composed chemically of single or multiple components within composite silica alcogels, aerogels and/or xerogels, and it is also suitable for continuous application.

1. Method for the preparation of silica alcogels, aerogels and xerogels, characterized in that
i) a reaction mixture is provided comprising at least the following:
silane reagent,
base catalyst,
gelation retarding additive,
aqueous/organic solvent mixture,
guest particle,
ii) the reaction mixture is agitated as necessary and sufficient until achieving the viscosity where spontaneous movement of the guest particles does not occur anymore; and
iii) the material obtained is shaped to a desired shape during or after step ii); then
iv) dried, if desired.

2. The method according to claim 1, wherein the gelation retarding reagent is a compound that does not react with the other components of the reaction mixture, and the molecules or molecular associations thereof are capable to form at least two hydrogen bonds at the same time.

3. The method according to claim 2, wherein there is at least two bridgehead atoms capable of forming hydrogen bonds in the molecule of the gelation retarding additive, wherein the distance between the closest bridgehead atoms is preferably up to 6 chemical bonds, as calculated on the shortest possible route on the covalent backbone of the molecule.

4. The method according to claim 3, wherein the gelation retarding additive is urea, dimethylformamide, dimethyl sulfoxide or a diol, such as ethylene glycol or propylene glycol or a polyol, such as glycerol or cellulose, or a mixture thereof.

5. The method according to claim 2, wherein there is one bridgehead atom capable of forming hydrogen bonds in the molecule of the gelation retarding additive, and the molecules form molecular associates that are capable of forming at least two hydrogen bonds.

6. The method according to claim 5, wherein the gelation retarding additive is pyridine.

7. The method according to claim 1, wherein the base catalyst is ammonia, ammonium carbonate, ammonium fluoride, hydrazine, hydroxylamine, or primary, secondary or tertiary amines, or a mixture thereof.

8. The method according to claim 1, wherein the gelation retarding additive plays the role of the catalyst as well, and is selected from the group consisting of the following: polyol amines, such as diethanolamine or triethanolamine, or di- or polyamines and amino alcohols, such as ethylenediamine, diethylenetriamine, diethanolamine, triethanolamine, piperazine, dibenzylamine, as well as diaza crown ethers containing two nitrogens and ether oxygens, for example 1,10-diaza-4,7,13,16-tetraoxacyclooctadecane, or a mixture thereof.

9. The method according to claim 1, wherein the silane reagent is selected from the group consisting of the following: alkoxysilanes, prehydrolized alkoxysilanes, open-chain or cyclic alkoxysilane oligomers, alkylalkoxysilanes, arylalkoxysilanes, arylalkylalkoxysilanes, glycidoxypropylalkoxysilanes, halogenoalkoxysilanes, halogeonalkylalkoxysilanes, vinylalkoxysilanes, alkenylalkoxysilanes, alkynylalkoxysilanes, as well as other substituted alkoxysilanes, including carbon chain substituted derivatives thereof, or a mixture thereof.

10. The method according to claim 9, wherein the silane reagent is tetramethoxysilane or tetraethoxysilane and the gelation retarding additive amounts to 1 to 50% of the reaction mixture.

11. (canceled)

12. The method according to claim 1, wherein the agitation of the mixture in step ii) is continued until reaching viscosity of about 2000 mPa·s and the aqueous/organic solvent mixture is an aqueous-alcoholic mixture.

13. (canceled)

14. The method according to claim 1, wherein the aqueous/organic solvent mixture is a methanol-water mixture and the co-solvent is ethanol, isopropanol, propanol, acetone, t-butanol, i-butanol, n-butanol, ethylene glycol, propylene glycol, dimethylformamide and/or dimethyl sulfoxide.

15. (canceled)

16. The method according to claim 1, wherein the guest particle is an element, alloy, an inorganic, organic, element-organic compound that does not react with the reaction mixture and is not or minimally soluble therein, composite, nanocrystal, nanorod, nanofilament, graphene, polymer, protein, enzyme, hormone, nucleic acid, fungus, spore, biological tissue, cell and/or virus, or a combination thereof.

17. The method according to claim 1, wherein in step ii), the agitation of the mixture is carried out continuously or intermittently, by shaking, rotating the reaction vessel, by mechanical or magnetic or magneto-hydrodynamic mixing of the mixture, by migration of electrically or magnetically charged particles, by flowing the reaction mixture, by passing through a liquid or gas and/or by ultrasonic treatment, or a combination of the processes listed.

18. The method according to claim 1, wherein in step iv) drying is carried out, if desired after a solvent exchange,
at room temperature and atmospheric pressure to obtain a xerogel, or
under supercritical conditions or by freeze-drying to obtain an aerogel.

19. Apparatus to achieve a method according to claim 1 in continuous manufacturing technology, characterized in that said apparatus is provided with a reagent vessel (1) for receiving a silane reagent or a solution thereof; a reagent vessel (2) for receiving a base catalyst or a solution thereof; a reaction chamber (3) connected to the reagent vessels (1,2); and a mixing device (4) having mixing elements positioned in the reaction chamber (3).

20. Apparatus according to claim 19, characterized in that said apparatus further comprises
a particle tank (5) for receiving at least one emulsion or suspension, connected to a mixing device (4b), and/or a macro chamber (6) for receiving macroparticles, and/or a gas-forming chamber (7) for receiving gas or gas-forming reagent(s),
wherein said reagent vessels (1,2) are connected to feeding devices (8a, 8b) connected to a mixing chamber (9) provided by a mixing device (4a),
wherein said particle tank (5), macro chamber (6) and the gas-forming chamber (7) are each connected to feeding means (8c, 8d, 8e) independently coupled either to said mixing chamber (9) provided with mixing means (4a), or to a second mixing chamber (9a) provided with mixing means (4d),
the mixing chamber (9) is connected to the reaction chamber (3) provided with the mixing device (4), the reaction chamber (3) is connected to a second mixing chamber (9a), and said second mixing chamber (9a) is connected to a second reaction chamber (3a) provided with a mixing means (4d).

21. Apparatus according to claim 19, characterized in that said reaction chamber (3) comprises a slightly tilted tubular reactor and scrapers are arranged in the vicinity of an inner surface of said reaction chamber (3).

22. Composite silica alcogel, aerogel or xerogel, obtainable by the method according to claim 1, and having guest particles with density below 0.98 g/cm³ dispersed therein.

23. Composite silica alcogel, aerogel or xerogel, obtainable by the method according to claim 1, and having guest particles with size over 1 mm dispersed therein.
